(19) United States
(12) Patent Application Publication
(10) Pub. No.: US 2003/0054371 A1
(43) Pub. Date: Mar. 20, 2003

(54) Polymorphic elements in the costimulatory receptor locus and uses thereof

(75) Inventors: Vincent Ling, Walpole, MA (US); Paul Wu, Cambridge, MA (US); Gary S. Gray, Brookline, MA (US)

Correspondence Address: LAHIVE & COCKFIELD, 28 STATE STREET, BOSTON, MA, 02109, US

(73) Assignee: Genetics Institute, Inc., Cambridge, MA (US)

(21) Appl. No.: 10/085,906

(22) Filed: Feb. 27, 2002

Related U.S. Application Data
(63) Continuation-in-part of application No. 09/534,061, filed on Mar. 24, 2000, now abandoned.
(60) Provisional application No. 60/126,215, filed on Mar. 25, 1999.

(51) Int. Cl. C12Q 1/68; C12P 19/34

(52) U.S. Cl. 435/6; 435/91.2

(57) ABSTRACT

The invention relates to polymorphic markers within the costimulatory receptor gene locus. These markers are characterized by sets of oligonucleotide primers according to the invention useful in PCR amplification and DNA segment resolution.

### CROSS-REFERENCE TO RELATED APPLICATIONS

This application claims priority to provisional application serial No. 60/126,215, entitled “Polymorphism of CTLA-4 and Uses Thereof,” filed on Mar. 25, 1999. This application is a continuation-in-part of U.S. Ser. No. 09/534,061, filed on Mar. 24, 2000, which corresponds to International Application Serial No. PCT/US00/07938 (Publication No. WO 00/56856) filed Mar. 24, 2000. The entire contents of these applications are incorporated herein by reference. Attached hereto is Appendix A containing materials related to this application. The entire contents of this appendix is hereby incorporated by reference.

### BACKGROUND OF THE INVENTION

In order for T cells to respond to foreign proteins, two signals must be provided by antigen-presenting cells (APCs) to resting T lymphocytes (Jenkins, M. and Schwartz, R. (1987) J. Exp. Med. 165, 302-319; Mueller, D. L., et al. (1990) J. Immunol. 144, 3701-3709). The first signal, which confers specificity to the immune response, is transduced via the T cell receptor (TCR) following recognition of foreign antigenic peptide presented in the context of the major histocompatibility complex (MHC). The second signal, termed costimulation, induces T cells to proliferate and become functional (Lenschow et al. 1996. Annu. Rev. Immunol. 14:233). Costimulation is neither antigen-specific, nor MHC restricted and is thought to be provided by one or more distinct cell surface molecules expressed by APCs (Jenkins, M. K., et al. 1988 J. Immunol 140, 3324-3330; Linsley, P. S., et al. 1991 J. Exp. Med. 173, 721-730; Gimmi, C. D., et al., 1991 Proc. Natl. Acad. Sci. USA. 88, 6575-6579; Young, J. W., et al. 1992 J. Clin. Invest. 90, 229-237; Koulova, L., et al. 1991 J. Exp. Med. 173, 759-762; Reiser, H., et al. 1992 Proc. Natl. Acad. Sci. USA. 89, 271-275; van-Seventer, G. A., et al. (1990) J. Immunol. 144, 4579-4586; LaSalle, J. M., et al., 1991 J. Immunol. 147, 774-80; Dustin, M. I., et al., 1989 J. Exp. Med. 169, 503; Armitage, R. J., et al. 1992 Nature 357, 80-82; Liu, Y., et al. 1992 J. Exp. Med. 175, 437-445).

The CD80 (B7-1) and CD86 (B7) proteins, expressed on APCs, are critical costimulatory molecules (Freeman et al. 1991. J. Exp. Med. 174:625; Freeman et al. 1989 J. Immunol. 143:2714; Azuma et al. 1993 Nature 366:76; Freeman et al. 1993. Science 262:909). B7 appears to play a predominant role during primary immune responses, while B7-1, which is upregulated later in the course of an immune response, may be important in prolonging primary T cell responses or costimulating secondary T cell responses (Bluestone. 1995. Immunity. 2:555).

One receptor to which B7-1 and B7 bind, CD28, is constitutively expressed on resting T cells and increases in expression after activation. After signaling through the T cell receptor, ligation of CD28 and transduction of a costimulatory signal induces T cells to proliferate and secrete IL-2 (Linsley, P. S., et al. 1991 J. Exp. Med. 173, 721-730; Gimmi, C. D., et al. 1991 Proc. Natl. Acad. Sci. USA. 88, 6575-6579; June, C. H., et al. 1990 Immunol. Today 11, 211-6; Harding, F. A., et al. 1992 Nature. 356, 607-609). A second receptor, termed CTLA4 (CD152) is homologous to CD28 but is not expressed on resting T cells and appears following T cell activation (Brunet, J. F., et al., 1987 Nature 328, 267-270). CTLA4 appears to be critical in negative regulation of T cell responses (Waterhouse et al. 1995. Science 270:985). Blockade of CTLA4 has been found to remove inhibitory signals, while aggregation of CTLA4 has been found to provide inhibitory signals that downregulate T cell responses (Allison and Krunimel. 1995. Science 270:932). In addition, lymphoproliferative disease has been associated with CTLA-4 gene-deficient mice (Bluestone, J. A., et al. (1997). J. Immunol 158: 1989-93; June et al., (1994) Immunol Today 15: 321-31; Tivol et al., (1996). Curr Opin Immunol 8:822-30; Tivol et al. (1995) Immunity 3: 541-7), although data conflicting this interpretation also exist (Liu, Y. (1997). Immunol Today 18: 569-72; Wu, Y. et al. (1997) J. Exp Med 185: 1327-35; Zheng, Y., et al. (1998) Proc Natl Acad Sci USA 95: 6284-9). Recently, a CD28-like receptor ICOS (Hutloff et al. 1999) and its B7-like cognate ligand, GL50 was identified in both mouse and humans systems (Ling et al. 2000 J Immunol 164: 1653-7; also known as B7RP or B7h, Yoshinaga, S. K., et al. 1999. Nature 402: 827; Swallow, M. M., et al. 1999 Immunity 11: 423). CD28 and ICOS exhibit protein sequence identity of ˜24%, just as the GL50 proteins also share ˜24% sequence identity with B7 proteins. Despite structural similarity, neither GL50 nor ICOS are likely to utilize the B7:CD28/CTLA4 costimulatory pathways because of the inability of GL50 to bind CD28/CTLA4 proteins and of the inability of B7 proteins to bind ICOS receptors (Ling, V., et al. 1999. Genomics 60: 341). In vitro analysis of ICOS mediated T-cell costimulation revealed that ICOS engagement resulted in enhanced T cell proliferation and Th-2 cytokine production. Blockade of the ICOS pathway by addition of ICOS-Ig to MLR (mixed lymphocyte reaction) or tetanus toxoid recall response assays resulted in decreased T-cell proliferation (Aicher, A., et al. 2000. J Immunol 164: 4689-96.). Transgenic mice expressing ICOS-ligand exhibited an increase in B-cell germinal center size and enhancement of immunoglobin production (Yoshinaga et al., supra) suggesting that overexpression of the ligand may influence B cell development. Taken together, these data are consistent with the model of the ICOS receptor serving as a pivotal signaling molecule involved with T-cell and B-cell proliferation and differentiation.

The genetic organization of CTLA-4 has been previously described (Brunet, J. F., et al., (1987). Nature 328: 267-70; Dariavach, P., et al., (1988). Eur J Immunol 18: 1901-5.) as being comprised of 4 exons which encode separate functional domains: a leader sequence, an extracellular domain, a transmembrane domain, and cytoplasmic domain. Within the extracellular domain, the B7 binding motif is centered on the amino acids MYPPPY, a sequence also found in the extracellular domain of CD28, the primary B7 receptor responsible for T-cell activation (Balzano, C., et al., (1992). Int J Cancer Suppl 7: 28-32). The cytoplasmic domain of CTLA-4 encodes the motif YVKM in which the phosphorylation state of tyrosine has been implicated in both signal transduction through SYP/SHP2 phosphatase (Marengere, L. E., et al., (1996). Science 272: 1170-3. [published errata appear in Science Dec. 6, 1996;274(5293)1597 and Apr. 4, 1997;276(5309):21]; Shiratori, T., et al (1997). Immunity 6: 583-9), and the intracellular accumulation of CTLA-4 via AP50 clatharin-mediated endocytosis (Chuang, E., et al., (1997). J. Immunol 159: 144-51; Zhang, Y., and Allison, J. P. (1997) Proc Natl Acad Sci USA 94: 9273-8). CTLA-4 has also been reported to be involved with T-cell receptor signaling by interfering with ERK and JNK activation (Calvo, C. R., et al., (1997). J Exp Med 186: 1645-53). Recently, polymorphisms in the non-coding region 3′ of human CTLA-4 DNA have been correlated with a number of autoimmune diseases, including: Grave's disease (Donner, H., et al., (1997a). J Clin Endocrinol Metab 82: 4130-2 Donner, H., et al., (1997b). J Clin Endocrinol Metab 82: 143-6; Kotsa, K., et al., (1997). Clin Endocrinol (Oxf) 46: 551-4; Nistico, L., et al., (1996). Hum Mol Genet 5: 1075-80), Hashimoto's disease (Braun, J., et al., (1998). Tissue Antigens 51: 563-6; Tomer, Y., et al., (1997). J Clin Endocrinol Metab 82: 1645-8, myasthenia gravis with thymoma (Huang, D., et al., (1998). J Neuroimmunol 88: 192-8), and IDDM (Marron, M. P., et al., (1997). Hum Mol Genet 6: 1275-82; Nistico, L., et al., (1996). Hum Mol Genet 5: 1075-80) in patients.

The minimal promoter of mouse CTLA-4 suggests that transcriptional initiation control is localized approximately 335 bp upstream from the initiation codon. However, the contribution from other regions of the CTLA-4 locus to the regulation of gene expression has not been examined (Finn, P. W., et al., (1997). J Immunol 158: 4074-81; Perkins, D., et al., (1996). J Immunol 156: 4154-9). Despite the tightly regulated control of CTLA-4 expression and the importance of this key immunoregulatory protein, the published genomic sequences of the human CTLA-4 are incomplete. Further, no data are available for the intron sequences of mouse CTLA-4. In addition, the genomic structure of other costimulatory receptors is not well understood.

Areas of simple repetitive DNA (i.e., microsatellite DNA) interspersed throughout the genome have been used extensively to map chromosomes. It has been found that these simple repeats often vary in length among individuals, thus, they have facilitated genetic linkage studies of diseases within populations. Unlike long and short interspersed repeats, the mechanism by which simple repeats are generated and inserted into the genome is not known, and their potential role in modulating biochemical processes is not clear (Epplen, C., et al., (1997). Electrophoresis 18: 1577-85; Epplen, J. T., et al., (1994). Biol Chem Hoppe Seyler 375: 795-801). In addition, single nucleotide polymorphisms (SNPs), resulting from variations, insertions, or deletions, result in base changes that contribute to the majority of phenotypic diversity.

Certain polymorphisms of a particular sequence in particular regions have been correlated with the development of, or susceptibility, to a disease or other condition. Because the genes responsible for disorders or conditions associated with the immune response have not all been cloned, it is useful to utilize such markers for a variety of diagnostic and prognostic assays. The utility of such markers depends upon how tightly the marker and the disease locus are linked. Accordingly, the identification of novel DNA polymorphisms that are associated with disease states is desirable and aids in the diagnosis or prognosis of diseases or conditions to which they are linked.

### SUMMARY OF THE INVENTION

This application relates, at least in part, to the identification of polymorphic elements, such as microsatellite repeat (“PMR”) or single nucleotide polymorphisms (“SNP”) sequences in the costimulatory receptor gene locus. These sequences are useful as markers e.g., identifying genetic material from a given individual and/or in identifying individuals at risk for developing a particular disease or condition or at risk for giving birth to an offspring likely to develop a particular disease or condition. In particular, the subject markers are linked to a variety of autoimmune diseases or conditions.

In one aspect, the invention pertains to a method for determining the predisposition of a human subject to develop autoimmune disease, said method comprising detecting a polymorphic microsatellite repeat (PMR) in the human costimulatory receptor gene locus, wherein the PMR sequence is not an hR2 sequence, to thereby determine the predisposition of a human subject to develop autoimmune disease.

In one embodiment, the PMR sequence selected from the group consisting of SEQ ID Nos.: 303, 306, 309, 312, 315, 321, 324, 327, 330, 333, 336, 339, 342, 345, 348, 351, 354, 357, 360, 363, 366, and 369.

In another embodiment, the autoimmune disease is selected from the group consisting of: insulin-dependent diabetes mellitus (IDDM), Addison's disease, Graves' disease, autoimmune hypothyroidism, myasthenia gravis, thymoma, lupus, thyroiditis, postpartum thyroiditis, rheumatoid arthritis, Hashimoto's disease, coeliac disease and leprosy.

In one embodiment, the step of detecting is performed using a polymerase chain reaction (PCR) employing a first and second primer.

In one embodiment, the first or second comprises the sequence selected from the group consisting of SEQ ID Nos.: 301, 302, 304, 305, 307, 308, 310, 311, 313, 314, 316, 317, 319, 320, 322, 323, 325, 326, 328, 329, 331, 332, 334, 335, 337, 338, 340, 341, 343, 344, 346, 347, 349, 350, 352, 353, 355, 356, 358, 359, 361, 362, 364, 365, 367, and 368.

In another aspect, the invention pertains to a method for determining the predisposition of a human subject to autoimmune disease, said method comprising detecting an hR1 PMR sequence to thereby determine the predisposition of a human subject to autoimmune disease.

In one embodiment, the autoimmune disease is selected from the group consisting of insulin-dependent diabetes mellitus (IDDM), Addison's disease, Graves' disease, autoimmune hypothyroidism, myasthenia gravis, thymoma, lupus, thyroiditis, postpartum thyroiditis, rheumatoid arthritis, Hashimoto's disease, coeliac disease and leprosy.

In one embodiment, the step of detecting is performed using PCR employing a first and second primer.

In another aspect, the invention pertains to a method for determining the polymorphic variant or subtype of a PMR sequence in the costimulatory receptor locus in a human subject, said method comprising detecting a polymorphic microsatellite repeat (PMR) in the human costimulatory receptor gene locus, wherein the PMR sequence is not an hR2 sequence to thereby determine the polymorphic variant or subtype of a PMR sequence in the costimulatory receptor locus in a human subject.

In one embodiment, the PMR sequence is selected from the group consisting of SEQ ID Nos.: 3, 6, 9, 12, 15, 18, 21, 24, 27, 30, 33, 36, 39, 42, 44, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, and 300.

In one embodiment, the step of detecting is performed using PCR employing a first and second primer.

In another aspect, the invention pertains to a PCR primer capable of amplifying a PMR sequence in the costimulatory receptor locus of a human subject, wherein the primer consists of a nucleotide sequence selected from the group consisting of: SEQ ID NO: 301, 302, 304, 305, 307, 308, 310, 311, 313, 314, 316, 317, 319, 320, 322, 323, 325, 326, 328, 32\9, 331, 332, 334, 335, 337, 338, 340, 341, 343, 344, 346, 347, 349, 350, 352, 353, 355, 356, 358, 359, 361, 362, 364, 365, 367, and 368.

In still another aspect, the invention pertains to a method for determining the predisposition of a human subject to develop autoimmune disease, said method comprising detecting single nucleotide polymorphism SNP) in the human costimulatory receptor gene, to thereby determine the predisposition of a human subject to develop autoimmune disease.

### BRIEF DESCRIPTION OF THE DRAWINGS

FIG. 1 is a sequence diagram of the human 2q33 costimulatory receptor region. The position of sequence line is indicated as nt. displayed. The stippled line represents human BAC clone 22700 sequence. Coding sequences of NADH: ubiquinone oxidoreductase, keratin-18 pseudogene, and nucleophosmin pseudogene, EST-like sequences, retroviral elements, CD28 (4 CDS), CTLA4 (4 CDS) and the ICOS (5 CDS) receptors are displayed as open boxes on the sequence line. Black bars beneath sequence line indicate regions of mouse sequence homology (>35 bp, >70% identity) based on limited sequencing of mouse BAC clone 23114 syntenic to human BAC clone 22700. White boxes below the sequence line indicate predicted ORFs by Grail; gray boxes indicate predicted ORFs by DiCTion. Sequences with homologies to Genbank STS and microsatellite repeats are marked as asterisks. Several of the polymorphic microsatellite repeats used in this study are indicated as SARA 43, SARA 1, SARA 31, CTLA4 3′ UTR, and SARA 47, referring to the first primer of the primer pair used to amplify them.

FIG. 2 panels A and B show hybridization analysis of 2q33 sequences. Panel A shows results of genomic microarray expression analysis of BAC clone 22700 sequences. Inserts from the sequenced BAC clone 22700 library were amplified and spotted onto glass slides. RNA probes were generated from either non-induced or PMA-ionomycin induced human CD4+ T-cells. Differential hybridization in 5/6 experiments yielded clones corresponding to those positions presented. Panel B shows identification of anti-sense ICOS transcripts. RNA blot of activated and non-activated RNA samples from two donor CD4+ T-cells preparation and Jurkat cell line were hybridized against strand-specific (either+or −) radiolabeled T7-transcripts of ICOS 340 -UTR region (right line drawing). ICOS 3′-UTR (−) probe hybridization reveals ICOS gene transcripts (left blot) while ICOS 3′ UTR (+) probe hybridization reveals LTR derived anti-sense-ICOS transcripts (right blot).

FIG. 3 shows identification of polymorphic microsatellite repeats within BAC clone 22700. Amplification of repeats amplified by SARA 31, CTLA4 3′ UTR, SARA 1, SARA 43, and SARA 47 followed by denaturing PAGE electrophoresis and autoradiography revealed polymorphic PCR products. Two alleles were detected in SARA 31 and CTLA4 3′ UTR; 4 alleles were detected in SARA 1, and >5 alleles were detected in both SARA 43 and SARA 47 amplification reactions.

FIG. 4 panels A, B, and C, show sequence alignment between mouse and human ICOS genomic DNA. Panel A shows GAP alignment of regions flanking CDS-1 (boxed) revealed two zones of sequence homology (as shown) separated by a ˜250 bp mouse-specific repetitive DNA region. Panel B shows dot plot alignment of human and mouse ICOS genomic regions including CDS-2 to CDS-5. Homologies greater than 60% identity over a 20 bp window are displayed. Panel C shows similarity plot of consensus sequence derived from GAP alignment between human and mouse ICOS genomic regions displayed in B. Breaks in similarity index indicates presence of non-conserved repetitive sequences. Aligned consensus coding sequences are indicated in top line while location of the conserved microsatellite repeat amplified by the SARA 47 primer set is denoted by an asterisk.

### DETAILED DESCRIPTION OF THE INVENTION

The instant invention provides polymorphic elements, e.g., polymorphic microsatellite repeat (“PMR”) or single nucleotide polymorphism (“SNP”) sequences in the costimulatory receptor gene locus. The invention also provides sequences that can be used to amplify PMR or SNP sequences. The polymorphic elements of the invention are useful as markers e.g., in genetic testing, for example, to identify genetic material from a given individual and/or in identifying individuals at risk for developing a particular disease or condition. In particular, the subject polymorphic elements are useful in identifying individuals that carry or are at risk for developing diseases or conditions associated with signaling via a costimulatory receptor, such as CD28, CTLA4, or ICOS, e.g., autoimmune diseases or conditions. Tables I and II list the sequences of PMRs of the invention and Table III lists the sequences comprising the SNPs of the invention (the SNP is shown in a bold uppercase letter).

I. Definitions

As used herein the term “costimulatory receptor gene locus” includes the genetic region comprising the genes encoding the costimulatory receptors CD28, CTLA4, and ICOS. This locus spans approximately 300 kb on chromosome 2q33.

As used herein the term “polymorphic microsatellite repeat (PMR)” includes regions of a chromosome containing runs of short repeated sequences (e.g., ATATAT). These simple microsatellite DNA repeats tend to be interspersed throughout the genome and the number of such repeats is highly variable in the population. For example, individuals may have a different number of copies of the repeat at a particular locus.

As used herein the term “polymorphism” with respect to a particular region of a DNA molecule includes naturally occurring variations in nucleotide sequence among individuals that occur in a particular region. Such polymorphisms can occur, e.g., when DNA from one individual has an insertion of an additional nucleotide(s), a deletion of a nucleotide(s), a substitution of a nucleotide(s) when compared to DNA from another individual. Polymorphisms in microsatellite repeats frequently lead to differences in the length of the repeat that can be easily visualized, e.g., by Southern blot analysis of chromosomal DNA fragments using an oligonucleotide probe to visualize the size DNA fragment containing the particular polymorphic element.

As used herein, the term “SNP” (single nucleotide polymorphism) includes polymorphisms in a single nucleotide, e.g., that occur when a nucleotide is changed, inserted, or deleted.

As used herein, the term “immune cell” includes cells that are of hematopoietic origin and that play a role in the immune response. Immune cells include lymphocytes, such as B cells and T cells; natural killer cells; myeloid cells, such as monocytes, macrophages, eosinophils, mast cells, basophils, and granulocytes.

As used herein, the term “costimulate” with reference to activated immune cells includes the ability of a costimulatory molecule to provide a second signal which is not transduced by an activating receptor (a “costimulatory signal”) that induces proliferation or effector function. For example, a costimulatory signal can result in cytokine secretion, e.g., in a T cell that has received a T cell-receptor-mediated signal. As used herein the term “costimulatory molecule” includes molecules which are present on antigen presenting cells (e.g., B7-1, B7, B7RP-1 (Yoshinaga et al. 1999. Nature 402:827), B7h (Swallow et al. 1999. Immunity. 11:423) and/or related molecules (e.g., homologs)) that bind to costimulatory receptors (e.g., CD28, CTLA4, ICOS (Hutloff et al. 1999. Nature 397:263), B7h ligand (Swallow et al. 1999. Immunity. 11:423) and/or related molecules) on T cells.

As used herein, the phrase “autoimmune disorder or condition” includes immune responses against self antigens. As used herein, the term “immune response” includes T and/or B cell responses, i.e., cellular and/or humoral immune responses.

As used herein, the term “detect” with respect to polymorphic elements includes various methods of analyzing for a polymorphism at a particular site in the genome. The term “detect” includes both “direct detection,” such as sequencing, and “indirect detection,” using methods such as amplification or hybridization.

II. Isolation of Genetic Material

The subject polymorphic elements are useful as markers, e.g., to identify genetic material as being derived from a particular individual or in making assessments regarding the propensity of an individual to develop a particular disorder or condition, the ability of an individual to respond to a certain course of treatment, or in other diagnostic or prognostic assays described in more detail below.

Genetic material suitable for use in such assays can be derived from a variety of sources. For example, nucleic acid molecules (preferably genomic DNA) can be isolated from a cell from a living or deceased individual using standard methods. Cells can be obtained from biological samples, e.g., from tissue samples or from bodily fluid samples that contain cells, such as blood, urine, semen, or saliva. The term “biological sample” is intended to include tissues, cells and biological fluids containing cells which are isolated from a subject, as well as tissues, cells and fluids present within a subject. The subject detection methods of the invention can be used to detect polymorphic elements in DNA in a biological sample in intact cells (e.g., using in situ hybridization) or in extracted DNA, e.g., using Southern blot hybridization. In one embodiment, immune cells are used to extract genetic material for use in the subject assays.

III. Polymorphic Elements in the Costimulatory Receptor Locus

Any of the PMRs or SNPs identified in the costimulatory receptor locus identified herein (see Tables I, II, and III of the application) can be utilized as a marker to detect DNA polymorphisms among individuals. Several approaches were taken to identify the subject polymorphic elements. In one approach, overlapping bacterial artificial chromosome (BAC) clones (clones 22700 and 22608) were isolated containing contiguous sequences corresponding to the costimulatory receptors in the order of: CD28, CTLA4, and ICOS. Shotgun sequencing of BAC clones in the region followed by gap closure, sequence alignment and assembly generated 381,403 base pairs of contiguous sequence containing all 3 receptors plus an endogenous HERV-H type endogenous retrovirus located 366 bp 3′ of ICOS in reverse orientation. A number of PMR sequences were identified in this contiguous sequence. In addition, the ICOS gene locus was localized to this region. In one 181 kb BAC clone containing both CTLA4 and ICOS genomic loci, the ICOS receptor was found to be encoded by 5 exons representing leader sequence, extracellular domain, transmembrane domain, cytoplasmic domain1 and cytoplasmic domain 2. Polymorphic elements identified in the costimulatory receptor locus (as well as exemplary primers that can be used to amplify them) are set forth in Tables I, II, and III.

In one embodiment, a polymorphic element of the invention is 5′ of the CD28 region. Polymorphic elements residing within nucleotides 243-41772 or the costimulatory receptor locus are 5′ of the CD28 region.

In one embodiment a PMR or SNP of the invention is in the CD28 region (e.g., the 5′UT, in an intron, or in the 3′ UT region of the CD28 gene) of the costimulatory receptor locus. Polymorphic elements residing within nucleotides 42348 and 73724 are within the CD28 region of the costimulatory receptor locus (see the start and end location of the subject PMR sequences and the location of the SNP sequences in Tables I, II, and III of the specification.) The polymorphic elements residing within nucleotides 73725 and 203643 are in the intergenic region between CD28 and CTLA4.

In one embodiment, the PMR sequence is in the CD28 gene and is selected from the group consisting of SEQ ID Nos.: 303, 306, 309, 312, 315, and 318 to thereby determine the predisposition of a human subject to develop autoimmune disease.

In one embodiment, the PMR sequence is in the CD28 gene and is selected from the group consisting of SEQ ID Nos.: 3, 6, 9, 12, 15, 18, 21, 24, 27, 30, 33, 36, 39, 42, 44, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, and 171 to thereby determine the predisposition of a human subject to develop autoimmune disease.

In another embodiment, a polymorphic element of the invention is in the CTLA4 region (e.g., the 5′ UT region, in an intron, or in the 3′UT region of the CTLA4 gene) of the costimulatory receptor locus. Preferably, where the polymorphic element is a polymorphic element in the CTLA4 region of the costimulatory receptor locus, the polymorphic element is not in the 3′ untranslated region of the CTLA4 gene. In another embodiment, a PMR of the invention is not hR2 and a primer that amplifies a polymorphic element in the CTLA4 region of the costimulatory receptor locus does not amplify an hR2 PMR sequence. PMRs and SNPs residing within nucleotides 203644 and 209793 are within the CTLA4 region of the costimulatory receptor locus (see the start and end location or positions of the subject polymorphic sequences in Tables I, II, and III of the specification.) The polymorphic elements residing within nucleotides 209792 and 272635 are in the intergenic region between CTLA4 and ICOS.

In one embodiment, the PMR sequence is in the CTLA4 gene and is selected from the group consisting of SEQ ID Nos.: 321, 324, 327, 330, 333, 336, 339, 342, 345, 348, 351, 354, and 357 to thereby determine the predisposition of a human subject to develop autoimmune disease.

In one embodiment, the PMR sequence is in the CTLA4 gene and is selected from the group consisting of SEQ ID Nos.: 174, 177, 180, 183, 186, 189, 192, 195, 198, 201, 204, 207, 210, 213, 216, 219, 222, 225, 228, 231, and 234 to thereby determine the predisposition of a human subject to develop autoimmune disease.

In one embodiment, a polymorphic element of the invention is in the ICOS region (e.g., the 5′UT, in an intron, or in the 3′ UT region of the ICOS gene) of the costimulatory receptor locus. PMRs or SNPs residing within nucleotides 272636 and 297393 are within the ICOS region of the costimulatory receptor locus (see the start and end location of the subject PMR and SNP sequences in Tables I, II, and III of the specification.)

In one embodiment, a polymorphic element of the invention is 3′ of the ICOS region. Polymorphic elements residing within nucleotides 300867-380660 are 3′ of the ICOS region.

In one embodiment, the PMR sequence is in the ICOS gene locus and is selected from the group consisting of: SEQ ID NO: 360:363, 366, and 369.

In one embodiment, the PMR sequence is in the ICOS gene locus and is selected from the group consisting of SEQ ID Nos.: 237, 240, 243, 246, 249, 252, 255, 258, 261, 264, 267, 270, 273, 276, 279, 282, 285, 288, 291, 294, 297, and 300.

IV. Polymorphic Elements in the Costimulatory Receptor Locus and Genetic Diseases

Polymorphisms in the CTLA-4 gene have been linked to various autoimmune diseases, such as insulin-dependent diabetes mellitus (IDDM) (Witas et al., Biomedical Letters 58: 163-168, 1998); Addison's disease, Graves' disease and autoimmune hypothyroidism (Kemp et al., Clin. Endocrinol. 49:609-613, 1998); myasthenia gravis and thymoma (Huang et al., J. Neuorimmunol. 88:192-198, 1998); lupus (Mehrian et al., Arthritis Rheum. 41:596-602, 1998); thyroiditis, particularly postpartum thyroiditis (Waterman et al., Clin. Endocrinol., 49:251-255, 1998); rheumatoid arthritis (Seidl et al., Tissue Antigens 51:62-66, 1998); Hashimoto's disease (Barbesino et al., J. Clin. Endocrnol. and Metab. 83:1580-1584, 1998); coeliac disease (Djilali-Saiah et al., Gut 43:187-189, 1998); and leprosy (Kaur et al., Hum. Genet. 100:43-50, 1997). Of these diseases, IDDM, Grave's disease and hypothyroidism (Kotsa, K., et al., (1997). Clin Endocrinol (Oxf) 46: 551-4; Marron, M. P., et al., (1997). Hum Mol Genet 6: 1275-82) have been found to be associated with certain alleles of the hR2 region of human CTLA-4. The PMR associated with the hR2 region of CTLA4 has the sequence: gttgtattgcatatatacatatatatatatatatatatatatatatatat (SEQ ID NO: 546). The PMR associated with the hR1 region of CTLA4 has the sequence: ctctccctt ctccctctct cccttcttct cttcctcttc cttctt (SEQ ID NO: 547)

Currently, there is no information available on whether the hR2 region confers biologically significant attenuation of CTLA-4 expression or whether this polymorphism is merely a marker for an associated gene closely linked to this CTLA-4 allele. The novel polymorphic elements described herein provide additional markers that may be more closely linked with certain autoimmune disorders or conditions. As described in the appended Examples, use of the instant polymorphic sequences as markers can provide different results, i.e., different distribution of polymorphisms, than those obtained using the hR2 marker, indicating that the polymorphic elements disclosed herein can be used to further refine genetic alleles linked to the costimulatory receptor locus. Exemplary polymorphic elements of the invention are shown in Tables I, II, and III.

V. Uses of Polymorphic Elements of the Invention

The polymorphic elements of the invention are useful as markers in a variety of different assays. The polymorphic elements of the invention can be used, e.g., in diagnostic assays, prognostic assays, and in monitoring clinical trials for the purposes of predicting outcomes of possible or ongoing therapeutic approaches. The results of such assays can, e.g., be used to prescribe a prophylactic course of treatment for an individual, to prescribe a course of therapy after onset of a disease or disorder, or to alter an ongoing therapeutic regimen.

Accordingly, one aspect of the present invention relates to diagnostic assays for detecting PMRs or SNPs in a biological sample (e.g., cells, fluid, or tissue) to thereby determine whether an individual is afflicted with a disease or disorder, or is at risk of developing a disorder linked to one or more of the subject polymorphisms. The subject assays can also be used to determine whether an individual is at risk for passing on the propensity to develop a disease or disorder to an offspring. The invention also provides for prognostic (or predictive) assays for determining whether an individual is at risk of developing a autoimmune disorder or condition. For example, polymorphisms in a PMR or SNP sequence can be assayed in a biological sample. Such assays can be used for prognostic, diagnostic, or predictive purpose to thereby phophylactically or therapeutically treat an individual prior to or after the onset of an autoimmune disorder associated with one or more polymorphisms.

In another embodiment, the methods further involve obtaining a control biological sample from a control subject, determining one or more polymorphic element in the sample and comparing the polymorphisms present in the control sample with those in a test sample.

The invention also encompasses kits for detecting the polymorphic elements in a biological sample. For example, the kit can comprise a primer capable of detecting one or more PMR and/or SNP sequences in a biological sample. The kit can further comprise instructions for using the kit to detect PMR and/or SNP sequences in the sample.

Polymorphisms in the costimulatory receptor locus among individuals can be used to identify genetic material as being derived from a particular individual. For example, minute biological samples can be obtained from an individual and an individual's genomic DNA can be amplified using primers which amplify one or more of the disclosed PMR sequences to obtain a unique pattern of bands. A particular band pattern can be compared with a band pattern in a sample known to have come from a certain individual to determine whether the patterns match. Other exemplary methods for detection are set forth below. Panels of corresponding DNA sequences from individuals can provide unique individual identifications, as each individual will have a unique set of such DNA sequences due to allelic differences.

The subject polymorphic elements can also be used in forensic biology. Forensic biology is a scientific field employing genetic typing of biological evidence found at a crime scene as a means for positively identifying, for example, a perpetrator of a crime. For example, to make such an identification, PCR technology can be used to amplify DNA sequences taken from very small biological samples found at a crime scene. The amplified sequence can then be compared to a standard, thereby allowing identification of the origin of the biological sample.

The polymorphic elements described herein can further be used to provide polynucleotide reagents, e.g., probes which can be used in, for example, an in situ hybridization technique, to identify a specific tissue, e.g., in cases where a forensic pathologist is presented with a tissue of unknown origin.

VI. Detection of Polymorphisms

Practical applications of techniques for identifying and detecting polymorphisms relate to many fields including forensic medicine, disease diagnosis and human genome mapping.

DNA polymorphisms can occur, e.g., when one nucleotide sequence comprises at least one of 1) a deletion of one or more nucleotides from a polymorphic sequence; 2) an addition of one or more nucleotides to a polymorphic sequence; 3) a substitution of one or more nucleotides of a polymorphic sequence, or 4) a chromosomal rearrangement of a polymorphic sequence as compared with another sequence. As described herein, there are a large number of assay techniques known in the art which can be used for detecting alterations in a polymorphic sequence.

Repeats associated with specific genetic alleles are commonly used as molecular markers in phenotyping human populations. Microsatellite repeats (simple repetitive elements) are defined as motifs of 1-6 bases in length and tandemly reiterated 5-100 times or more. The assay of repeats is amenable to automation, and thus has gained wide use in forensic science and genetic disease linkage determination. These repeats are dispersed throughout the genome and currently are not known to have any definitive biological function, although some reports suggest a role of microsatellites in binding nuclear proteins. Indeed a growing number of genetic diseases are being attributed to the presence of alleles containing unusually large repeats (Epplen, C., et al., (1997). Electrophoresis 18: 1577-85).

Analysis of polymorphisms is amenable to highly sensitive PCR approaches using specific primers flanking the repetitive sequence of interest. In one embodiment, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting polymorphisms in the PMR sequence (see Abravaya et al. (1995) Nucleic Acids Res .23:675-682).

This method can include the steps of collecting a sample of cells from a patient, isolating nucleic acid (e.g., genomic, DNA) from the cells of the sample, contacting the nucleic acid sample with one or more primers which specifically amplify a PMR sequence under conditions such that hybridization and amplification of the PMR sequence (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting polymorphisms described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et all, 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, after extraction of genomic DNA, amplification is performed using standard PCR methods, followed by molecular size analysis of the amplified product (Tautz, 1993; Vogel, 1997). Typically DNA amplification products are labeled by the incorporation of radiolabelled nucleotides or phosphate end groups followed by fractionation on sequencing gels alongside standard dideoxy DNA sequencing ladders. By autoradiography, the size of the repeated sequence can be visualized and detected heterogeneity in alleles recorded. More recent innovations include the incorporation of fluorescently labeled nucleotides in PCR reactions followed by automated sequencing. Both methods have been used in the study of a human CTLA-4 repeats (Yanagawa, T., et al., (1995). J Clin Endocrinol Metab 80: 41-5 Huang, D., et al., (1998). J Neuroimmunol 88: 192-8.

In other embodiments, polymorphisms can be identified by hybridizing a sample and control nucleic acids to high density arrays containing hundreds or thousands of oligonucleotides probes (Cronin, M. T. et al. (1996) Human Mutation 7: 244-255; Kozal, M. J. et al. (1996) Nature Medicine 2: 753-759). For example, polymorphisms can be identified in two dimensional arrays containing light-generated DNA probes as described in Cronin, M. T. et al. supra. Briefly, a first hybridization array of probes can be used to scan through long stretches of DNA in a sample and control to identify base changes between the sequences by making linear arrays of sequential overlapping probes. This step allows the identification of polymorphisms. This step is followed by a second hybridization array that allows the characterization of specific polymorphisms by using smaller, specialized probe arrays complementary to all polymorphisms detected.

At the present time in this art, the most accurate and informative way to compare DNA segments requires a method which provides the complete nucleotide sequence for each DNA segment. Particular techniques have been developed for determining actual sequences in order to study polymorphism in human genes. See, for example, Proc. Natl. Acad. Sci. U.S.A. 85, 544-548 (1988) and Nature 330, 384-386 (1987); Maxim and Gilbert. 1977. PNAS 74:560; Sanger 1977. PNAS 74:5463. In addition, any of a variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol 38:147-159).

In genetic mapping, the most frequently used screening for DNA polymorphisms arising from mutations consist of digesting the DNA strand with restriction endonucleases and analyzing the resulting fragments by means of Southern blots. See Am. J. Hum. Genet. 32, 314-331 (1980) or Sci. Am. 258, 40-48 (1988). Since polymorphisms often occur randomly they may affect the recognition sequence of the endonuclease and preclude the enzymatic cleavage at that cite.

Restriction fragment length polymorphism mappings (RFLPS) are based on changes at a restriction enzyme site. In one embodiment, polymorphisms from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Moreover, the use of sequence specific ribozymes (see, for example, U.S. Pat. No. 5,498,531) can be used to score for the presence of a specific ribozyme cleavage site.

Another technique for detecting specific polymorphisms in particular DNA segment involves hybridizing DNA segments which are being analyzed (target DNA) with a complimentary, labeled oligonucleotide probe. See Nucl. Acids Res. 9, 879-894 (1981). Since DNA duplexes containing even a single base pair mismatch exhibit high thermal instability, the differential melting temperature can be used to distinguish target DNAs that are perfectly complimentary to the probe from target DNAs that only differ by a single nucleotide. This method has been adapted to detect the presence or absence of a specific restriction site, U.S. Pat. No. 4,683,194. The method involves using an end-labeled oligonucleotide probe spanning a restriction site which is hybridized to a target DNA. The hybridized duplex of DNA is then incubated with the restriction enzyme appropriate for that site. Reformed restriction sites will be cleaved by digestion in the pair of duplexes between the probe and target by using the restriction endonuclease. The specific restriction site is present in the target DNA if shortened probe molecules are detected.

Other methods for detecting polymorphisms in nucleic acid sequences include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the polymorphic sequence with potentially polymorphic RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels. See, for example, Cotton et al. (1988) Proc. Natl Acad Sci USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping polymorphisms obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a polymorphic sequence is hybridized to a DNA molecule from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify polymorphisms. For example, single strand conformation polymorphism (SSCP) may be used to detect differences in electrophoretic mobility between mutant and wild type nucleic acids (Orita et al. (1989) Proc Natl. Acad. Sci USA: 86:2766, see also Cotton (1993) Mutat Res 285:125-144; and Hayashi (1992) Genet Anal Tech Appl 9:73-79). Single-stranded DNA fragments of sample and control PMR nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of nucleic acid molecule comprising polymorphic sequences in polyacrylamide gels containing a gradient of denaturant is assayed using denaturing gradient gel electrophoresis (DGGE) (Myers et al. (1985) Nature 313:495). When DGGE is used as the method of analysis, DNA can be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys Chem 265:12753).

Examples of other techniques for detecting polymorphisms include, but are not limited to, selective oligonucleotide hybridization, selective amplification, or selective primer extension. For example, oligonucleotide primers may be prepared in which the polymorphic region is placed centrally and then hybridized to target DNA under conditions which permit hybridization only if a perfect match is found (Saiki et al. (1986) Nature 324:163); Saiki et al. (1989) Proc. Natl Acad. Sci USA 86:6230). Such allele specific oligonucleotides are hybridized to PCR amplified target DNA or a number of different polymorphisms when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the polymorphism of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the polymorphic region to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known polymorphism at a specific site by looking for the presence or absence of amplification.

Another process for studying differences in DNA structure is the primer extension process which consists of hybridizing a labeled oligonucleotide primer to a template RNA or DNA and then using a DNA polymerase and deoxynucleoside triphosphates to extend the primer to the 5′ end of the template. Resolution of the labeled primer extension product is then done by fractionating on the basis of size, e.g., by electrophoresis via a denaturing polyacrylamide gel. This process is often used to compare homologous DNA segments and to detect differences due to nucleotide insertion or deletion. Differences due to nucleotide substitution are not detected since size is the sole criterion used to characterize the primer extension product.

Another process exploits the fact that the incorporation of some nucleotide analogs into DNA causes an incremental shift of mobility when the DNA is subjected to a size fractionation process, such as electrophoresis. Nucleotide analogs can be used to identify changes since they can cause an electrophoretic mobility shift. See, U.S. Pat. No. 4,879,214.

The use of certain nucleotide repeat polymorphisms for identifying or comparing DNA segments have been described (e.g., by Weber & May 1989. Am Hum Genet 44:388; Litt & Luthy. 1989 Am Hum Genet 44:397).

Many other techniques for identifying and detecting polymorphisms are known to those skilled in the art, including those described in “DNA Markers: Protocols, Applications and Overview,” G. Caetano-Anolles and P. Gresshoff ed., (Wiley-VCH, New York) 1997, which is incorporated herein by reference as if fully set forth.

Since a polymorphic marker and an index locus occur as a “pair”, attaching a primer oligonucleotide according to the present invention to one member of the pair, e.g., the polymorphic marker allows PCR amplification of the segment pair. The amplified DNA segment can then be resolved by electrophoresis and autoradiography. A resulting autoradiograph can then be analyzed for its similarity to another DNA segment by autoradiography. Following the PCR amplification procedure, electrophoretic mobility enhancing DNA analogs may optionally be used to increase the accuracy of the electrophoresis step.

In addition, many approaches have also been used to specifically detect SNPs. Such techniques are known in the art and many are described e.g., in DNA Markers: Protocols, Applications, and Overviews. 1997. Caetano-Anolles and Gresshoff, Eds. Wiley-VCH, New York, pp 199-211 and the references contained therein). For example, in one embodiment, a solid phase approach to detecting polymorphisms such as SNPs can be used. For example an oligonucleotide ligation assay (OLA) can be used. This assay is based on the ability of DNA ligase to distinguish single nucleotide differences at positions complementary to the termini of co-terminal probing oligonucleotides (see, e.g., Nickerson et al. 1990. Proc. Natl. Acad. Sci. USA 87:8923. A modification of this approach, termed coupled amplification and oligonucleotide ligation (CAL) analysis, has been used for multiplexed genetic typing (see, e.g., Eggerding 1995 PCR Methods Appl. 4:337); Eggerding et al. 1995 Hum. Mutat. 5:153).

In another embodiment, genetic bit analysis (GBA) can be used to detect a SNP of the invention (see, e.g., Nikiforov et al. 1994. Nucleic Acids Res. 22:4167; Nikiforov et al. 1994. PCR Methods Appl. 3:285; Nikiforov et al. 1995. Anal Biochem. 227:201). In another embodiment, microchip electrophoresis can be used for high-speed SNP detection (see e.g., Schmalzing et al. 2000. Nucleic Acids Research, 28). In another embodiment, matrix-assisted laser desorption/ionization time-of-flight mass (MALDI TOF) mass spectrometry can be used to detect SNPs (see, e.g., Stoerker et al. Nature Biotechnology 18:1213).

In one embodiment of the invention, more than one polymorphism (e.g., more than one PMR, more than one SNP, and/or at least one PMR and at least one SNP) may be detected to enhance the ability of a particular polymorphic profile to be correlated with the presence or absence of a disorder or the propensity to develop a disorder.

The methods described herein may be performed, for example, by utilizing pre-packaged diagnostic kits comprising at least one probe/primer nucleic acid or antibody reagent described herein, which may be conveniently used, e.g., in clinical settings to diagnose patients exhibiting symptoms or family history of a disease or illness involving a polymorphic elements. In addition, a readily available commercial service can be used to analyze samples for the polymorphic elements of the invention.

VII. Primers for Amplification of Polymorphic Elements

Given the discovery of the instant polymorphic elements, primers can readily be designed to amplify the polymorphic sequences by one of ordinary skill in the art. For example, a PMR or SNP sequence of the invention can be identified in GenBank Accession Numbers AF411059 (BAC 22608), AF411058 (BAC 22700) or AF411057 (BAC 22606) or used for homology searching of another database containing human genomic sequences (e.g., using Blast or another program) and the location of the PMR or SNP sequence and/or flanking sequences can be determined and the appropriate primers identified. For example, using the flanking sequences one of ordinary skill in the art could readily identify a primer for use in amplifying a PMR sequence of the invention.

In another embodiment a primer of the invention amplifies a PMR or SNP in the CD28 region (e.g., the 5′UT, in an intron, or in the 3′ UT region of the CD28 gene) of the costimulatory receptor locus.

In one embodiment, a first or second primer detects a gene in the CD28 locus and comprises the sequence selected from the group consisting of SEQ ID Nos.: 301, 302, 304, 305, 307, 308, 310, 311, 313, 314, 316, and 317.

In another embodiment, a primer of the invention amplifies a PMR or SNP in the CTLA4 region (e.g., the 5′ UT region, in an intron, or in the 3′UT region of the CTLA4 gene) of the costimulatory receptor locus. Preferably, where the primer amplifies a PMR in the CTLA4 region of the costimulatory receptor locus, the PMR is not in the 3′ untranslated region of the CTLA4 gene. In another embodiment, a PMR primer of the invention that amplifies a PMR in the CTLA4 region of the costimulatory receptor locus does not amplify an hR2 PMR sequence.

In one embodiment, a first or second primer detects a gene in the CTLA4 locus and comprises or consists of the sequence selected from the group consisting of SEQ ID Nos.: 319, 320, 322, 323, 325, 326, 328, 329, 331, 332, 334, 335, 337, 338, 340, 341, 343, 344, 346, 347, 349, 350, 352, 353, 355, and 356.

In another aspect, the invention is directed to a PCR primer capable of amplifying a PMR sequence in the costimulatory receptor locus of a human subject, wherein the primer comprises or consists of a nucleotide sequence selected from the group consisting of: SEQ ID NO: 301, 302, 304, 305, 307, 308, 310, 311, 313, 314, 316, 317, 319, 320, 322, 323, 325, 326, 328, 329, 331, 332, 334, 335, 337, 338, 340, 341, 343, 344, 346, 347, 349, 350, 352, 353, 355, 356, 358, 359, 361, 362, 364, 365, 367, and 368.

In one embodiment, a PMR primer of the invention amplifies a PMR in the ICOS region (e.g., the 5′UT, in an intron, or in the 3′ UT region of the ICOS gene) of the costimulatory receptor locus.

In one embodiment, a first or second primer detects a gene in the ICOS locus and comprises the sequence selected from the group consisting of SEQ ID Nos.: 358, 359, 361, 362, 364, 365, 367, and 368.

In one embodiment, a primer for amplification of a polymorphic elements is at least about 5-10 base pairs in length. In one embodiment, a primer for amplification of a polymorphic elements is at least about 15-20 base pairs in length. In one embodiment, a primer for amplification of a polymorphic elements is at least about 20-30 base pairs in length. In one embodiment, a primer for amplification of a polymorphic elements is at least about 30-40 base pairs in length. In one embodiment, a primer for amplification of a polymorphic elements is at least about 40-50 base pairs in length. In one embodiment, a primer for amplification of a polymorphic elements is at least about 50-60 base pairs in length. In one embodiment, a primer for amplification of a polymorphic elements is at least about 60-70 base pairs in length. In one embodiment, a primer for amplification of a polymorphic elements is at least about 70-80 base pairs in length. In one embodiment, a primer for amplification of a polymorphic elements is at least about 80-90 base pairs in length. In one embodiment, a primer for amplification of a polymorphic elements is at least about 90-100 base pairs in length. In one embodiment, a primer for amplification of a polymorphic elements is at least about 100-110 base pairs in length. In one embodiment, a primer for amplification of a polymorphic elements is at least about 110-120 base pairs in length. In one embodiment, a primer for amplification of a polymorphic elements is at least about 120-130 base pairs in length. In one embodiment, a primer for amplification of a polymorphic elements is at least about 130-140 base pairs in length. In one embodiment, a primer for amplification of a polymorphic elements is at least about 140-150 base pairs in length. In one embodiment, a primer for amplification of a polymorphic elements is at least about 150-160 base pairs in length. In one embodiment, a primer for amplification of a polymorphic elements is at least about 160-170 base pairs in length. In one embodiment, a primer for amplification of a polymorphic elements is at least about 170-180 base pairs in length. In one embodiment, a primer for amplification of a polymorphic elements is at least about 180-190 base pairs in length. In one embodiment, a primer for amplification of a polymorphic elements is at least about 190-200 base pairs in length.

In one embodiment, a primer for amplification of a PMR sequence of the invention is located at least about 200 base pairs away from (upstream or downstream of) the PMR sequence to be amplified (i.e., leaving about 200 nucleotides from the end of the primer sequence to the PMR). In another embodiment, a primer for amplification of a PMR sequence of the invention is located at least about 150 base pairs away from (upstream or downstream of) the PMR sequence to be amplified. In another embodiment, a primer for amplification of a PMR sequence of the invention is located at least about 100 base pairs away from (upstream or downstream of) the PMR sequence to be amplified. In another embodiment, a primer for amplification of a PMR sequence of the invention is located at least about 75 base pairs away from (upstream or downstream of) the PMR sequence to be amplified. In another embodiment, a primer for amplification of a PMR sequence of the invention is located at least about 50 base pairs away from (upstream or downstream of) the PMR sequence to be amplified. In another embodiment, a primer for amplification of a PMR sequence of the invention is located at least about 25 base pairs away from (upstream or downstream of) the PMR sequence to be amplified. In another embodiment, a primer for amplification of a PMR sequence of the invention is located at least about 10 base pairs away from (upstream or downstream of) the PMR sequence to be amplified. In another embodiment, a primer for amplification of a PMR sequence of the invention is located at least about 5 base pairs away from (upstream or downstream of) the PMR sequence to be amplified. In yet another embodiment a primer for amplification of a PMR sequence of the invention is adjacent to the PMR sequence to be amplified.

Preferred primers for amplification of a PMR sequence of the invention include the SARA primer pairs set forth in Table II of the specification.

In one embodiment, a primer for the amplification of a PMR sequence comprises a nucleotide sequence selected from the group consisting of: SARA 41, SARA 42, SARA 43, SARA 44, SARA 45, SARA 46, SARA 17, SARA 18, SARA 19, SARA 20, SARA 25, SARA 26, SARA 1, SARA 2, SARA 3, SARA 4, SARA 39, SARA 40, SARA 33, SARA 34, SARA 35, SARA 36, SARA 37, SARA 38, SARA 11, SARA 12, SARA 13, SARA 14, SARA 21, SARA 22, SARA 23, SARA 24, SARA 9, SARA 10, SARA 31, SARA 32, SARA 5, SARA 6, SARA 7, SARA 8, SARA 27, SARA 28, SARA 29, SARA 30, SARA 47, and SARA 48.

In one embodiment, SARA 43 primer is not used to detect a PMR of the invention. In another embodiment, when a SARA 43 primer is used to detect a PMR, it is used in combination with a primer detecting a second, different PMR.

In one embodiment, more than one PMR can be detected, e.g., in a multiplex assay. For example, two sets of primer pairs are used to detect two PMRs. Preferably, when more than one PMR is detected, the PMRs are about 50 kb in distance from each other. For instance, in one example, the SARA primer pairs 47 and 48 are used to detect a first PMR and the SARA primer pairs 1 and 2 are used to detect a second PMR. In another embodiment three different sets of primer pairs are used to detect three PMRs. In yet another embodiment, four different sets of primer pairs are used to detect four PMRs. For example, the SARA primer pairs 31 and 30, 1 and 2, 43 and 44, and 47 and 48 are used in combination to detect four PMRs.

VIII. Detecting Differentially Transcribed Genes in Genomic DNA

The instant invention also provides methods of detecting differential transcription of genes in genomic DNA samples. According to the methods, genomic DNA is subcloned using methods and vectors known in the art, e.g., BAC vectors. Genomic DNA is used to make arrays. Methods of making genomic DNA arrays are known in the art and can be found, e.g., in Lashkari et al. 1997. PNAS 94:13057; DeRisi et al. 1997. Science. 278:680; Ramsay 1998 Nature Biotechnology 16: 40; Wodicka et al. 1997. Nature Biotechnology 15:1359; Marshall and Hodgson. 1998. Nature Biotechnology 16: 27; Shoemaker et al. Nature. 2001. 409:922 and U.S. Pat. No. 5,807,222. The prior art methods of generating genomic microarrays have relied on finding open reading frames and amplifying them. However, there can be mistakes in computer generated open reading frames. In the instant invention, rather than selecting open reading frames for amplification, randomly picked vectors are used as templates for amplification, e.g., by PCR, using standard methods such as M13 primers. Thus, the arrays of the instant invention are not based on selecting open reading frames prior to making the arrays. The products of PCR amplification are analyzed for the presence of a single band and are purified using standard methods. PCR products are arrayed onto a solid surface, e.g., slides.

Arrays can then be probed using standard methods, for example, total RNA can be prepared from stimulated or unstimulated cells. Probes can be prepared by including a label, e.g., dCTP in a cDNA synthesis reaction.

Hybridization can be performed under standard conditions, e.g., at 42° C. for 16 h in a buffer containing 50% formamide, 5×SSC, 0.1% SDS and DNA, e.g., salmon sperm DNA or human COT-1 DNA. The arrays can be washed using standard methods, e.g., in 1×SSC, 0.2% SDS for 5 min, and twice in 0.1×SSC, 0.2% SDS for 10 min and then rinsed in water and dried.

Scanning can be carried out using a commercially available system and the data quantitated.

Using the disclosed methods or variations thereof it is possible to determine not only those genes that are differentially transcribed, but the relative position of the genes in the genome. In one embodiment, this information can be used in a transcription profiling method that examines the correlation between expression patterns of transcribed DNA and loci attributed to genetic diseases. Using such a method, when a disease has been shown to be linked to a particular marker, but it is not known exactly what gene is responsible for the disease, differential regulation of genes in the region of the marker can be examined. In another embodiment, RNA isolated from disease and control samples can be used as probes to determine whether altered transcription levels of gene products exist between the disease and control samples. Because the instant genomic arrays contain positional information, in one embodiment, it is possible to experimentally identify genomic regions bordering transcription initiation, intron/exon boundaries and regions downstream of transcriptional response elements located near a gene. In yet another embodiment, the instant methods can be used to uncover novel genes or transcriptional control elements to which genetic associations are mapped.

The contents of all references, pending patent applications and published patents, cited throughout this application are hereby expressly incorporated by reference. Each reference disclosed herein is incorporated by reference herein in its entirety. Any patent application to which this application claims priority is also incorporated by reference herein in its entirety.

TABLE 1

| 5′ Flanking sequence | 3′ Flanking sequence | Start | End | PMR SEQUENCE |
|---|---|---|---|---|
| gcaggtggag (SEQ ID NO:1) | aattcttcca (SEQ ID NO:2) | Start: 198 | End: 223 | tttttttcttttt (SEQ ID NO:3) |
| gatgaggctgagaatttgca | aaaaaaaaaccgtaatacat | | | |
| cctataaaga (SEQ ID NO:4) | gagacagagt (SEQ ID NO:5) | Start: 1183 | End: 1212 | attttatttattta (SEQ ID NO:6) |
| ttaccctgagaactaatgag | cttgctccgtcggccaggct | | | ttatttatttattttt |
| cagcctggac (SEQ ID NO:7) | ttagaactgg (SEQ ID NO:8) | Start: 2117 | End: 2154 | aaaaaaaaaaatat (SEQ ID NO:9) |
| aacagagcgagactccatct | atcttcctaggtttattggt | | | atatatatatatatatatatatat |
| gggtatatt (SEQ ID NO:10) | acatgctgg (SEQ ID NO:11) | Start: 4676 | End: 4799 | ttttatgttttttc (SEQ ID NO:12) |
| tatatcaccatttgaaagaaa | ccgggtggggtggctgacacc | | | tttgttttaaaaatttacatttttttctt |
| | | | | attctttatttaaagaaatttttctttgt |
| | | | | tttaaaaatttacatttttttcttattct |
| | | | | ttatttgttaccttaaaaatttta |
| aatacttga (SEQ ID NO:13) | gggacagag (SEQ ID NO:14) | Start: 6350 | End: 6393 | attttacattttgt (SEQ ID NO:15) |
| aatgtgtgattgaagaattga | tcttgctctgttgcccaggca | | | tggattttattttattttattttattttt |
| tatttaata (SEQ ID NO:16) | gagggttgc (SEQ ID NO:17) | Start: 8996 | End: 9050 | ttttcttttctttt (SEQ ID NO:18) |
| cacttttacgaagtccccatg | aaggtttattgtgaagagtga | | | ttcttttctttcctttttttttttttttt |
| | | | | ttttttt |
| gtaatctta (SEQ ID NO:19) | aaacagata (SEQ ID NO:20) | | | |
| ctttctcatgtataaagatac | catagtttatatgatattaaa | Start: 13241 | End: 13264 | atatatatatatat (SEQ ID NO:21) |
| | | | | tatatatata |
| acaggcaaa (SEQ ID NO:22) | gagccggag (SEQ ID NO:23) | Start: 16951 | End: 16982 | tttttgtttttgtt (SEQ ID NO:24) |
| aagagaacaaccaacaccctg | tctcgctctgtcaccaggctg | | | tttgtttttgtttttttt |
| tgttaagat (SEQ ID NO:25) | aacattata (SEQ ID NO:26) | Start: 18993 | End: 19023 | gtgtgtgtgtgtgt (SEQ ID NO:27) |
| tttattgtcctgtaccctgta | tatcagccaccacacatgtac | | | gtgtgtgtgtgtgtgtg |
| ttccagtat (SEQ ID NO:28) | ggtagatga (SEQ ID NO:29) | Start: 23525 | End: 23577 | agtgtgcgtgtgtg (SEQ ID NO:30) |
| tgagaccatagggaatgcagt | gatgctgatgggaaccggata | | | tgtttgtgagtgtgtatgtgtgtgtgcca |
| | | | | tccatgtgtg |
| gacctggtt (SEQ ID NO:31) | agccaatgg (SEQ ID NO:32) | Start: 31546 | End: 31565 | gtgtgtgtgtgtgt (SEQ ID NO:33) |
| tagatgggtcagaagtgggga | tggctggagacagtatctttg | | | gtgtgt |
| attattttt (SEQ ID NO:34) | cctccaatg (SEQ ID NO:35) | Start: 32828 | End: 32926 | gtatgtgtgtatgc (SEQ ID NO:36) |
| ttggctctgtattattccatg | tatagctatagcccatattct | | | gtatgtgtatgtgtgtgtgtgtatacatt |
| | | | | ccttttgtacgtgtgtgtgtgtgtgtgtg |
| | | | | tgtgtgttatatatatatataatacata |
| gcctgggca (SEQ ID NO:37) | tatggcaca (SEQ ID NO:38) | Start: 36676 | End: 36719 | aaaaaataaaaaaa (SEQ ID NO:39) |
| acaagagcgaaactctgtctc | tatatactatggaatactatg | | | taaaattaaattaaattaaaaaaaagaaa |
| | | | | a |
| atgaaaatc (SEQ ID NO:40) | cattctgtt (SEQ ID NO:41) | Start: 41617 | End: 41902 | ttctttctttctct (SEQ ID NO:42) |
| tctcctctgctagagacttta | gccctggctggagtgcagtgg | | | ctctcccccttccttccttccttccctcc |
| | | | | ctccctctttctttctttccatctttctt |
| | | | | tctttctttctttctttctttctttcttt |
| | | | | ctttctttctttctttctttctttctttc |
| | | | | tttctctttctttctttcttttctttctt |
| | | | | tctttctttctctttctctctctttcttt |
| | | | | cttcctttcttttttctctctccccttcc |
| | | | | ttccttctttccttcttttcttttctttt |
| | | | | cttttctttctttctctctttctttctgt |
| | | | | ctttcttttct |
| ctctagcta (SEQ ID NO:43) | aattgagat (SEQ ID NO:44) | Start: 42872 | End: 42896 | atttattattatta (SEQ ID NO:45) |
| ttagttgatagtgtcccaaga | ggggtttcactatgttgccca | | | ttattatttta |
| ctgagaaac (SEQ ID NO:46) | aagaatagt (SEQ ID NO:47) | Start: 45156 | End: 45198 | atgtgtatctgtgt (SEQ ID NO:48) |
| cactgttatgcctgtgttgag | tcttttttccattaatttaat | | | gcgtgcatgtgtgtgtatgtatgtatatg |
| gccgttttt (SEQ ID NO:49) | aagacaatt (SEQ ID NO:50) | Start: 46986 | End: 47011 | accaaaaacccaaa (SEQ ID NO:51) |
| ggccaatgacagggtgttagc | cactcagagctttgagcctga | | | accaaaaaaacc |
| gacatgtgg (SEQ ID NO:52) | agcattgca (SEQ ID NO:53) | Start: 49710 | End: 49779 | gagagagagagaga (SEQ ID NO:54) |
| aaggagccaagtgctggacct | gggcttgagggtagagaaggg | | | gagggagagagagagagagagagacagag |
| | | | | agagagagtgtgtgtgtgtgtgtgtg |
| tgtgtgtgt (SEQ ID NO:55) | gatttgttg (SEQ ID NO:56) | Start: 49795 | End: 49888 | gagggtagagaagg (SEQ ID NO:57) |
| gtgtagcattgcagggctt | cataggagatgagtgtattag | | | gtattattaggaaagaaaggaggaggagg |
| | | | | gagaggaaaaaaagagggtggggatagtt |
| | | | | ttctcaaggagatagggaggga |
| ttagaactg (SEQ ID NO:58) | ttggatgtt (SEQ ID NO:59) | Start: 58175 | End: 58218 | aaaaaaaggaaaca (SEQ ID NO:60) |
| acctaatgactccttctaagt | tgtattaagacaggtcgaact | | | aacaaaataaatcaaaaacaaaaaaacaa |
| | | | | a |
| tgtgctcca (SEQ ID NO:61) | gagacggac (SEQ ID NO:62) | Start: 63287 | End: 63389 | cctttccattttct (SEQ ID NO:63) |
| taatcttcctctgtaaaagta | tctcgctctgtcgcccaggct | | | ttttccttccttccttccttccttccttc |
| | | | | cttccttccttccttccttccttccttcc |
| | | | | ttttcttttctttttctttttcttttttt |
| | | | | tt |
| ggactacag (SEQ ID NO:64) | gagagggag (SEQ ID NO:65) | Start: 63536 | End: 63589 | ttattttttgtata (SEQ ID NO:66) |
| gtgccgccaccacgcctggc | tcttgctctgtcgcccaggct | | | tttatttatttatttattttaattaatta |
| | | | | atttttttttt |
| cagcctggg (SEQ ID NO:67) | cacaaaatt (SEQ ID NO:68) | Start: 71898 | End: 71933 | caaacaacaacaac (SEQ ID NO:69) |
| cgacagagtgagactccatct | atttgagtactgtgaaggatt | | | aacaacaacaacaacaacaaac |
| tactcatat (SEQ ID NO:70) | ccctatcat (SEQ ID NO:71) | Start: 75368 | End: 75420 | cacacacacacaca (SEQ ID NO:72) |
| catagctgaacactctaatag | cttctgggtaggggaagggaa | | | cacacacacacacacacagacacacacac |
| | | | | acacacacacac |
| cactgaagg (SEQ ID NO:73) | ggaggccct (SEQ ID NO:74) | Start: 77153 | End: 77189 | aaacaaagagaaag (SEQ ID NO:75) |
| atatgtgtgggtgtcacctga | gtttcggaaagaagagccagt | | | acagagagagaaaaagaagaaaa |
| atgataggc (SEQ ID NO:76) | agtagagat (SEQ ID NO:77) | Start: 78676 | End: 78702 | atttttttattttt (SEQ ID NO:78) |
| acacgccaccactcccggcta | ggggtttcaccatgttggcca | | | tatttttattttt |
| tattcagtg (SEQ ID NO:79) | acttcctgc (SEQ ID NO:80) | Start: 80664 | End: 80702 | ttctctctctttct (SEQ ID NO:81) |
| cctcccttctcccctgcctat | catgtgatctttacataccag | | | cactctttctctctctctatctctc |
| ttgagctgc (SEQ ID NO:82) | agttcttgt (SEQ ID NO:83) | Start: 82441 | End: 82495 | tccttgcttccttc (SEQ ID NO:84) |
| agattgagccgacttgaattc | catgtgagacagtaaataact | | | cttccttccttccttccttccttcctccc |
| | | | | ttccttccttcc |
| tttggggct (SEQ ID NO:85) | cctataatc (SEQ ID NO:86) | Start: 85097 | End: 85152 | ctaataaaatatat (SEQ ID NO:87) |
| tatcccaagggcaagggaaag | ttacattgcagaaagctataa | | | aattttaaaaatttgcttaaaattattat |
| | | | | ttataatatataa |
| gccattctc (SEQ ID NO:88) | ggccaggcg (SEQ ID NO:89) | Start: 88168 | End: 88203 | aaaaaacaaaacaa (SEQ ID NO:90) |
| ataggctttcctttgataatt | tggtggctcatgcctgtaatc | | | aacaaaacaaaaaacattaaaa |
| tgttctcaa (SEQ ID NO:91) | ttgtatagc (SEQ ID NO:92) | Start: 101708 | End: 101737 | gtgtgtgtgtgtgt (SEQ ID NO:93) |
| ggacaaaaggtttaatctcta | cacatcaagcatgatatcgtt | | | gtgtgtgtgtgtgtgt |
| aacatatat (SEQ ID NO:94) | gagacagaa (SEQ ID NO:95) | Start: 101998 | End: 102037 | tctttctttcttt (SEQ ID NO:96) |
| tcttcatagatacagaaaaca | tcttactctgttgcccaggct | | | ttcttttttcttttttctttttctttc |
| tccatcctg (SEQ ID NO:97) | ttggtgaag (SEQ ID NO:98) | Start: 106399 | End: 106439 | caaaaaacaaaca (SEQ ID NO:99) |
| ggcgacagagagactccgtct | acgatagatctcaagtgtttg | | | aacaaacaaacaaacaaacaaaaaacaa |
| tatagatt (SEQ ID NO:100) | gcagcaag (SEQ ID NO:101) | Start: 108817 | End: 108883 | ctcatcatcaaaa (SEQ ID NO:102) |
| actcgtgcttttcttcagcttc | ttttccttttttctgtggaacc | | | tcattatcattttcatcatcatcatcatc |
| | | | | atcatcatcatcatcatcttcatca |
| ccgggcga (SEQ ID NO:103) | aagaagtg (SEQ ID NO:104) | Start: 113754 | End: 11378 | cagggggcagggg (SEQ ID NO:105) |
| cacagcgagcgagacttcatct | agttctacacaaataaataaat | | | gcagggcggggagggg |
| cattccat (SEQ ID NO:106) | gaaacttc (SEQ ID NO:107) | Start: 118012 | End: 118042 | taaaaataaaata (SEQ ID NO:108) |
| atttttaccacctgaatttttg | tggggacattgaaagagtcagt | Start: 125017 | End: 125041 | aataaaataatttaaaaa |
| | | | | SARA41;SARA41/42 |
| cggctggc (SEQ ID NO:109) | tttttgag (SEQ ID NO:110) | Start: 125017 | End: 125041 | tatatatatacat (SEQ ID NO:111) |
| tggatgacttgaccatttacat | atggagtcttgccgtgttgccc | Start: 125845 | End: 125892 | atatatatatat |
| | | | | SARA 43;SARA43/44 |
| atctgctt (SEQ ID NO:112) | ctatgttt (SEQ ID NO:113) | Start: 125845 | End: 125892 | gtgtgtgtgtgtg (SEQ ID NO:114) |
| ttctatttctcctctttcactg | atttcaggtcatgatctgcttc | | | tgtgtgtgtgtgtgtgtgtgtgtgtgtgt |
| | | | | gtgtgtgt |
| aaatccta (SEQ ID NO:115) | aaaagtac (SEQ ID NO:116) | Start: 130753 | End: 130809 | atatctgtgtata (SEQ ID NO:117) |
| ccatttatctgatgatttatga | agaagggcacacactggttgtt | | | tatgtatatgcataaatatgcttgtatgt |
| | | | | ggctatgtgtatata |
| agcctgaa (SEQ ID NO:118) | gagacagg (SEQ ID NO:119) | Start: 133882 | End: 133915 | tttattattatta (SEQ ID NO:120) |
| acaaggacttggatgcaggcag | gtctcactctgtcacccagact | | | ttattttattattattttttt |
| gcctgggc (SEQ ID NO:121) | tgttgaag (SEQ ID NO:122) | Start: 135534 | End: 135560 | aaaaaaaataaaa (SEQ ID NO:123) |
| aacaagagtgaaactccatctc | gagtgacccaacatacacacag | | | taaaataagtaaaa |
| gacttcca (SEQ ID NO:124) | gagacgga (SEQ ID NO:125) | Start: 136451 | End: 136484 | aatttctattatt (SEQ ID NO:126) |
| gcttccagaactgtgagaaata | gtcttgctctgtcgcccaggct | | | tatttatttatttattttttt |
| gatacaga (SEQ ID NO:127) | atgggagg (SEQ ID NO:128) | Start: 139189 | End: 139251 | gtgtgtttggtgt (SEQ ID NO:129) |
| gtttagtgtggctgggtaagga | caggggaaggggagattaggga | Start: 139206 | End: 139251 | ggctgtgtgtgtgtgtgtgtgtgtgtgtg |
| | | Start: 143199 | End: 143252 | tgtgtgtgtctgtgtgtgtgt |
| | | | | PW210;PW210/211 |
| | | | | SARA45;SARA45/46 |
| aagggggg (SEQ ID NO:130) | atgcaaaa (SEQ ID NO:131) | Start: 143201 | End: 143252 | aggaggaagagag (SEQ ID NO:132) |
| gacaggcaaatgacgtattaga | atatgctggaataaaattgcta | | | gcagagagagagaaagggagagagatggg |
| | | | | gagagagagag |
| atcctttc (SEQ ID NO:133) | acatattg (SEQ ID NO:134) | Start: 146978 | End: 146995 | gtgtgcgtgtgtg (SEQ ID NO:135) |
| agagacagtacaatggtgttga | tacaggtaggtattacatatgt | Start: 146984 | End: 147075 | tgtgt |
| | | Start: 150056 | End: 150091 | SARA17;SARA17/18 |
| | | | | SARA19;SARA19/20 |
| gacatttt (SEQ ID NO:136) | gagacaga (SEQ ID NO:137) | Start: 150056 | End: 150091 | ttctctctctttc (SEQ ID NO:138) |
| gattatacctaagaaatggaaa | gtctcactctgtcacccagact | | | tctctctcttttttttcttcttt |
| gtccttat (SEQ ID NO:139) | gagagaaa (SEQ ID NO:140) | Start: 160968 | End: 160988 | tctctctctctctc (SEQ ID NO:141) |
| aagaaaaggaagagataccagg | agtccaagtgaggacatagcaa | | | tgtctct |
| tgtttgcc (SEQ ID NO:142) | gtagagac (SEQ ID NO:143) | Start: 164066 | End: 164091 | atttttatttttat (SEQ ID NO:144) |
| accatgcccagctaattaaatt | agagtctcatcatgttgcccag | | | tttttatttttt |
| agcctggg (SEQ ID NO:145) | gtctgtat (SEQ ID NO:146) | Start: 164828 | End: 164855 | aaaaataaataaat (SEQ ID NO:147) |
| cgacagagcaagactcagtctc | cttaattacatctgcaaagtcc | | | aaataaataaaaaa |
| gatgatgc (SEQ ID NO:148) | ggcttagc (SEQ ID NO:149) | Start: 178882 | End: 178908 | tttctttccttttt (SEQ ID NO:150) |
| aaatctagaaatgagaagtatg | aaagccacagaaactctaggtt | | | ttcccctctttt |
| aattaata (SEQ ID NO:151) | aattgtga (SEQ ID NO:152) | Start: 181152 | End: 181178 | tttcttttttaaaa (SEQ ID NO:153) |
| ttaagataaaacctgggaccag | tggatacatactagttttacat | | | ttttaaaattttt |
| ccatccct (SEQ ID NO:154) | aaggcgga (SEQ ID NO:155) | Start: 182110 | End: 182150 | ctttcttttctttt (SEQ ID NO:156) |
| gtattcgtggtgcagttaaaaa | gtcttgctctgtcgcccaggct | | | cttttcttttctttttttttttttttt |
| agtaactt (SEQ ID NO:157) | cctttctt (SEQ ID NO:158) | Start: 188627 | End: 188808 | attcttgcctttcc (SEQ ID NO:159) |
| gctctgagaatgcatgaactta | tcttttttggccccactgactc | | | ttctttccttcttttccttccctccttcct |
| | | | | tccttccttctttccttccttccttccttc |
| | | | | ctccccctccctcccttcctccctcttccc |
| | | | | tcctctctctctttctcgcctttctctctc |
| | | | | tctccccctttctctctctccccctttctc |
| | | | | tctctctcctctc |
| | | Start: 189057 | End: 189081 | SARA25;SARA25/26 |
| tagagtaa (SEQ ID NO:160) | ttttttaa (SEQ ID NO:161) | Start: 189057 | End: 189082 | atatatatatatat (SEQ ID NO:162) |
| tttctgggtttttagatttgga | cagtgtccactcactgctcaga | | | atatatatatat |
| ttttctaa (SEQ ID NO:163) | tatgtaag (SEQ ID NO:164) | Start: 192034 | End: 192092 | tcataaaaatggaa (SEQ ID NO:165) |
| ccacaaagtaacacatagacat | catcactaagatgttactatat | | | aatacagataggtaaaataagaaaataaaa |
| | | | | attttatgtaaaaat |
| ttgaatac (SEQ ID NO:166) | tcttcttt (SEQ ID NO:167) | Start: 193089 | End: 193152 | agtttctgttttgt (SEQ ID NO:168) |
| atgcaaattatccttcatttaa | actctcattctcttaaaataaa | | | ctttcttcctttgtttcatttgtttgtcat |
| | | | | ttctcttattcttctcattt |
| tgtacaaa (SEQ ID NO:169) | tttgagac (SEQ ID NO:170) | Start: 196402 | End: 196427 | ttattattattatt (SEQ ID NO:171) |
| tcgctttgtgaccacagttaac | gtctcgctctgttgcccaggct | | | attattattatt |
| ctctgcct (SEQ ID NO:172) | tatacaca (SEQ ID NO:173) | Start: 208025 | End: 208110 | actctcccttctcc (SEQ ID NO:174) |
| aaggccagctttgccattgcaa | tacacaaagatatactctattc | | | ctctctcccttcttctcttcctcttccttc |
| | | | | ttctcgctctttctctctctctctttctcc |
| | | | | ctctctgtctctctctttctctctctcttt |
| | | | | ctccctctctgtctct |
| tttagcca (SEQ ID NO:175) | tttaattt (SEQ ID NO:176) | Start: 209177 | End: 209216 | atatatacatatat (SEQ ID NO:177) |
| gtgatgctaaaggttgtattgc | gatagtattgtgcatagagcca | Start: 209177 | End: 209216 | atatatatatatatatatatatatat |
| | | | | Yanagawa-CTLA4 3′UTR |
| aagttttg (SEQ ID NO:178) | ccccagca (SEQ ID NO:179) | Start: 210625 | End: 210699 | tgtctgtgtctctt (SEQ ID NO:180) |
| agaatcactgcttaggcaactc | agtgctaacaaacacacaagac | | | cctctgtctctcccccttgctcattctctt |
| | | | | gcttgctcttgctctctttctctctttctt |
| | | | | g |
| tccactct (SEQ ID NO:181) | ggcaccaa (SEQ ID NO:182) | Start: 216200 | End: 216232 | gtgtgtgtgtggtg (SEQ ID NO:183) |
| gaatcgctgggggtggaggcag | gcaggggtgaaagctgattatg | | | tgtgtgtggtgtgtgtgtg |
| catgcggg (SEQ ID NO:184) | cattcgtt (SEQ ID NO:185) | Start: 217439 | End: 217489 | ttatatctatctat (SEQ ID NO:186) |
| ttaatacttaataaacacccct | ctgtccctctagagaaccctga | Start: 217444 | End: 217492 | ctatctatctatctatctatctatctatct |
| | | | | atc |
| | | | | SARA1;SARA1/2 |
| tgttttcc (SEQ ID NO:187) | agacaaag (SEQ ID NO:188) | Start: 219182 | End: 219215 | tgtttttgtttgtt (SEQ ID NO:189) |
| tgtgcatagatttacaagtcta | tcttgctctgtcgcccaggctg | Start: 219183 | End: 219214 | tgtttgtctgtttgtttttg |
| | | | | SARA3;SARA3/4 |
| aagtgctt (SEQ ID NO:190) | ctttttga (SEQ ID NO:191) | Start: 220194 | End: 220253 | aatactatataata (SEQ ID NO:192) |
| agaatgggcctgacacagggta | cccctcatagtatttggttcag | Start: 229431 | End: 229467 | ttgatatatgtggtaagtatattactatag |
| | | | | tatatttttattattt |
| | | | | SARA39;SARA39/40 |
| gtggcccc (SEQ ID NO:193) | aaaattat (SEQ ID NO:194) | Start: 229431 | End: 229467 | gagagaaagagaa (SEQ ID NO:195) |
| acagaccctatccttctggctt | agccagtggctgctgcaaatcc | | | gcaaagcagagagagagagagaga |
| ctgggtga (SEQ ID NO:196) | aaaaactg (SEQ ID NO:197) | Start: 230748 | End: 230810 | acacacacacaca (SEQ ID NO:198) |
| cagagtgagaccctgtctgaaa | ctgcttgggtcccaacatagac | Start: 230749 | End: 230810 | cacacacacacacacactacacacacaca |
| | | | | catccccacacaacaacaca |
| | | | | SARA33;SARA33/34 |
| cttgagta (SEQ ID NO:199) | gccttttt (SEQ ID NO:200) | Start: 231617 | End: 231659 | agaaaaaaaaaag (SEQ ID NO:201) |
| ctcaggtgcttcaaggttattc | atgtttttctatctttttttct | Start: 231619 | End: 231709 | agagagagaaaacagaaaaaagaataaaa |
| | | | | a |
| | | | | SARA35;SARA35/36 |
| gagagaga (SEQ ID NO:202) | gaatgctg (SEQ ID NO:203) | Start: 231661 | End: 231709 | cctttttatgttt (SEQ ID NO:204) |
| aaacagaaaaaagaataaaaag | aaggaaagtattatggtcactg | Start: 234817 | End: 234857 | ttctatctttttttctctctttcctctct |
| | | | | gctttct |
| | | | | SARA37;SARA37/38 |
| tgtatgag (SEQ ID NO:205) | gattctgt (SEQ ID NO:206) | Start: 234821 | End: 234844 | tctctctcttact (SEQ ID NO:207) |
| ccaattctgtataataaatctg | ttccccaaagaaccctgactaa | | | ccctctctctc |
| ttttgaca (SEQ ID NO:208) | atgccatt (SEQ ID NO:209) | Start: 239361 | End: 239421 | taaatttatcatg (SEQ ID NO:210) |
| ttaaacatgttccttctttcta | acttggtaaagtgaaagctaac | | | gtagtttatattaatatctttattatttt |
| | | | | taaaaatttatttatttta |
| gtatcttt (SEQ ID NO:211) | agcttgtc (SEQ ID NO:212) | Start: 239615 | End: 239634 | ctttttctctttt (SEQ ID NO:213) |
| actacttcctagtcttaccttc | tccttaactcttttgttatgaa | Start: 243340 | End: 243365 | tcttttt |
| | | Start: 245299 | End: 245342 | SARA11;SARA11/12 |
| | | | | SARA13;SARA13/14 |
| ctcacttc (SEQ ID NO:214) | gataaaaa (SEQ ID NO:215) | Start: 245299 | End: 245342 | acacacacacaca (SEQ ID NO:216) |
| cttccttctaaagcaaagctat | taaaaatgcagctttccaggca | | | cacacgcacacacacacgcacacacacac |
| | | | | ac |
| tttgctct (SEQ ID NO:217) | ccttgtcc (SEQ ID NO:218) | Start: 245540 | End: 245557 | aaaaaattttaaa (SEQ ID NO:219) |
| aagcctgtgcataaaactgttt | cttacactgcattgcacgcatg | Start: 249355 | End: 249387 | aaaaa |
| | | Start: 249821 | End: 249860 | SARA21;SARA21/22 |
| | | Start: 253000 | End: 253044 | SARA23;SARA23/24 |
| | | | | SARA9;SARA9/10 |
| tctctctg (SEQ ID NO:220) | actaattt (SEQ ID NO:221) | Start: 253030 | End: 253044 | tgtgtgtgtgtgt (SEQ ID NO:222) |
| tgttgttcacatgatctctctc | tttcttataaggacacctgtca | Start: 263177 | End: 263211 | gt |
| | | | | SARA31;SARA31/32 |
| ctcaagtg (SEQ ID NO:223) | gagatgga (SEQ ID NO:224) | Start: 263177 | End: 263213 | attttatttttat (SEQ ID NO:227) |
| gttcaacacttaagaatggggaca | gtctctcgctctgtcgctcagg | | | ttttatttttatttttattttttt |
| acattctg (SEQ ID NO:226) | aaacatta (SEQ ID NO:227) | Start: 264580 | End: 26463 | tatatattgtttt (SEQ ID NO:228) |
| gtgcctatcctttccctttttc | agcttatatatatgctgtacat | | | tgtatatttttctttatagtatttttata |
| | | | | taaaattttt |
| ctaaaaca (SEQ ID NO:229) | gtatgtat (SEQ ID NO:230) | Start: 265832 | End: 265847 | atatatatatata (SEQ ID NO:231) |
| aaggtctttgaattttatgtgc | gtactgccaattgagtgtcatc | Start: 265833 | End: 265858 | tat |
| | | | | SARA5;SARA5/6 |
| tattccct (SEQ ID NO:232) | attctagc (SEQ ID NO:233) | Start: 266114 | End: 266161 | tgtgtgtgtgtgt (SEQ ID NO:234) |
| agcggcaatgtacagctgaagc | tggttataaactgtagagaagc | | | gtgtgtgtgtgtgtgtgtgtgtgtgtatg |
| | | | | tgtgtg |
| aaaacatt (SEQ ID NO:235) | ttttgaga (SEQ ID NO:236) | Start: 283137 | End: 283164 | tattataaaaatt (SEQ ID NO:237) |
| agtctattgatatcaacagtaa | cggagtcttgctctgttgccag | | | attattattattatt |
| gggacctg (SEQ ID NO:238) | atgtctca (SEQ ID NO:239) | Start: 288676 | End: 288724 | gagtttgtttttg (SEQ ID NO:240) |
| ataattcaggtatataaatcat | ttatttttgtgatgtgtttgcc | | | tagtttgttgtttctgttgttcccttgtt |
| | | | | ttcttgt |
| gcctgggg (SEQ ID NO:241) | tctggggc (SEQ ID NO:242) | Start: 290427 | End: 290473 | gaaaagaaaagaa (SEQ ID NO:243) |
| aacagagtaaacccttttctct | tttatcttattatcaggccttg | | | aagaaaagaaagagagagaaaaagtaaac |
| | | | | aaaaa |
| gtctgttc (SEQ ID NO:244) | caattatt (SEQ ID NO:245) | | | acatatattttaatatataatatgtaatatacatacatatataaaatat |
| ttttactctttagcaggaggac | catttttaaactacttcgtact | Start: 290594 | End: 290746 | ataatgtatgtgt (SEQ ID NO:246) |
| | | | | gtatatatgtatgtatatgtatacaatta |
| | | | | tttgtatatatacactcacatagtctcta |
| | | | | tatattgtaatatacatatacatataata |
| | | | | tata |
| ggtgttga (SEQ ID NO:247) | atgaaagg (SEQ ID NO:248) | Start: 295266 | End: 295317 | gtgtgtgtgtgag (SEQ ID NO:249) |
| agcataaagatgagtttgcatg | caatggagaggggaaagcttct | | | tgtgtgtgtgtgtgtgtgcacgtgtgtgt |
| | | | | tgtgtgt |
| tagcctgg (SEQ ID NO:250) | ccaacgaa (SEQ ID NO:251) | Start: 313426 | End: 313471 | caaacataaataa (SEQ ID NO:252) |
| gcaacagaatgagactccatct | aaggaatggaagcaaatagcag | | | ataagtaaataaataaataaataaacaaa |
| | | | | caaa |
| gagagaca (SEQ ID NO:253) | agggtact (SEQ ID NO:254) | Start: 314103 | End: 314148 | aaaataaaataaa (SEQ ID NO:255) |
| ttacatagatccttcagacacc | gtattagtcagagttctctaga | | | ataaaataaaataaaataaaataaaataaaa |
| | | | | ta |
| actggtat (SEQ ID NO:256) | cagccaac (SEQ ID NO:257) | Start: 316798 | End: 316812 | atatatatatata (SEQ ID NO:258) |
| gtatacaattcacaaaagagag | aaacatgaaaaagttgtttcct | | | ta |
| caggaagg (SEQ ID NO:259) | gagatgga (SEQ ID NO:260) | Start: 321979 | End: 322027 | tatttatttattt (SEQ ID NO:261) |
| aaagcttgtagccacagaaagc | gtcttgcgctattgccacactg | | | atttatttatttatttatttatttattta |
| | | | | tttattt |
| gagcttgc (SEQ ID NO:262) | ggcctatg (SEQ ID NO:263) | Start: 331764 | End: 331799 | aagtgagtgagtg (SEQ ID NO:264) |
| aggaatgaaagctgatctgggt | acattactgtatactactgtag | | | agtgagtggtgagtgaaggtgag |
| atattctt (SEQ ID NO:265) | ggtgaaaa (SEQ ID NO:266) | Start: 332230 | End: 332276 | atttaacattttt (SEQ ID NO:267) |
| attccatacactttttcctatg | actaagacagaaacacacatta | | | atttattaattaatttttactttttaaac |
| | | | | tattt |
| tctgcaca (SEQ ID NO:268) | cattttag (SEQ ID NO:269) | Start: 340041 | End: 340077 | tcagatcaatcaa (SEQ ID NO:270) |
| aagcaggagctccaagagctat | tagattgaagtttaatatgctt | | | tcaatcaatcaacctatcaatcaa |
| ggtattca (SEQ ID NO:271) | tcgcccag (SEQ ID NO:272) | Start: 340210 | End: 340370 | ggaaaatgcgaaa (SEQ ID NO:273) |
| caggggttagattatacattat | aacaagaagagtttgtagacta | | | gagaaagaagagaaagaagatgaggaaag |
| | | | | agaaaaagaaagaaagaaaaaagaaagag |
| | | | | aaagaaaaaaagaaaacaaaagaaagaaa |
| | | | | ggaaggaaggaagaaaggaaggaaggaag |
| | | | | gaaggaaagaaggaaacaagggaagagaa |
| | | | | aaa |
| caaattga (SEQ ID NO:274) | agacagac (SEQ ID NO:275) | Start: 349153 | End: 349216 | gaaagagaagcag (SEQ ID NO:276) |
| gagaaacgtttatagaaagaaa | ctagacttaatgactgcattta | | | aggtgagagagggagggagaaaaagagaa |
| | | | | gaaaagaaatcccaagagagag |
| cagagaga (SEQ ID NO:277) | ttaaaatc (SEQ ID NO:278) | Start: 357553 | End: 357567 | tctctctctctct (SEQ ID NO:279) |
| tgattaaagaataacactagat | gctaacccactgtcatatcttt | | | ct |
| aatgatat (SEQ ID NO:280) | atcttagt (SEQ ID NO:281) | Start: 361537 | End: 361595 | gctttcctccatc (SEQ ID NO:282) |
| tgtcattttactctaaaattat | aacaactgtagtcatcattgtt | | | tcatttttttcactttttccctcttctgt |
| | | | | ctcttctctttttcttt |
| aattttaa (SEQ ID NO:283) | agatggag (SEQ ID NO:284) | Start: 363349 | End: 363386 | attttttgtttgt (SEQ ID NO:285) |
| acaactggatcttctgggcaac | tctcactctgtcatccaggctg | | | ttgtttgtttatctgtttctttttg |
| atgcacat (SEQ ID NO:286) | ctgctgtt (SEQ ID NO:287) | Start: 372334 | End: 372378 | attttttgtttgt (SEQ ID NO:288) |
| gtaccctagaatttaaagtatt | ttccaaagaggttgcaccattt | | | ttgtttgtttatctgtttctttttggaaa |
| tttctatt (SEQ ID NO:289) | gaagaaag (SEQ ID NO:290) | Start: 374670 | End: 374720 | aaaaaagaaaaga (SEQ ID NO:291) |
| gcttgggataggctattcaagt | accaacacagacactctgaaaa | | | aaagaaaagaaaaggaaagaaaagaaaaa |
| | | | | aggagacagaaaaggaggaggaggaggaa |
| | | | | aatggggagaaggagaaggag |
| ccagccgg (SEQ ID NO:292) | ccccactg (SEQ ID NO:293) | Start: 377126 | End: 377161 | caaaaacaaaaaa (SEQ ID NO:294) |
| gacaacagtgcgagactccatc | tactagagcaatcataaggact | | | acaaaacaaaacaaaacaaaaaa |
| attccatt (SEQ ID NO:295) | atgtatct (SEQ ID NO:296) | Start: 377473 | End: 377523 | ttgtgtgtgtgtg (SEQ ID NO:297) |
| tttgtgttttcctaaggacact | gtggcttatccgagcttctaga | | | tgagtgtgcgtgcacacgtgtgtgcacgt |
| | | | | gtgtgtgtg |
| atgacaga (SEQ ID NO:298) | gagacgga (SEQ ID NO:299) | Start: 379698 | End: 379729 | tttttcttttctt (SEQ ID NO:300) |
| gacagcactatgtttatccaag | gtcttgctctgtcccccaggct | | | ttcttttcttttttttttt |

TABLE II

| | | | | PMR |
|---|---|---|---|---|
| SARA PRIMER PAIRS | | Start | End | SEQUENCE |
| SARA 41 (SEQ ID NO:301) | SARA 42 (SEQ ID NO:302) | 125017 | 125041 | tatatatatacatat (SEQ ID NO:303) |
| GCTGGCTG | CCACTGCA | | | atatatatat |
| GATGACTTG | CTCCAGCCT | | | (SEQ ID NO:303) |
| ACC | GGG | | | |
| SARA 43 (SEQ ID NO:304) | SARA 44 (SEQ ID NO:305) | 125845 | 125892 | gtgtgtgtgtgtgt (SEQ ID NO:306) |
| TATTTCTCC | TGACCTGAA | | | gtgtgtgtgtgtgt (SEQ ID NO:306) |
| TCTTTCACT | ATAAACATA | | | gtgtgtgtgtgtgt |
| GG | GA | | | gtgtgt |
| SARA 45 (SEQ ID NO:307) | SARA 46 (SEQ ID NO:308) | 143199 | 143252 | gaaggaggaaga (SEQ ID NO:309) |
| GGGGGGAC | TATTCCAGC | | | gaggcagagaga |
| AGGCAAAT | ATATTTTTG | | | gagaaagggaga |
| GACG | CA | | | gagatggggaga |
| | | | | gagaga |
| SARA 17 (SEQ ID NO:310) | SARA 18 (SEQ ID NO:311) | 146984 | 147075 | gtgtgtgtgtgtac (SEQ ID NO:312) |
| GAGACAGT | ATGTAAAAA | | | atattgtacaggta |
| ACAATGGTG | CATAAATAT | | | ggtattacatatgt |
| TTG | GTATGTG | | | atacatattacacg |
| | | | | tacagttaatatata |
| | | | | tgtgtatgtatgtgt |
| | | | | gtacac |
| SARA 19 (SEQ ID NO:313) | SARA 20 (SEQ ID NO:314) | 150056 | 150091 | ttctctctctttctct (SEQ ID NO:315) |
| TGATTATAC | CCACTACAC | | | ctctcttttttttcttc |
| CTAAGAAAT | TCTAGTCTG | | | ttt |
| GG | GG | | | |
| SARA 25 (SEQ ID NO:316) | SARA 26 (SEQ ID NO:317) | 189057 | 189081 | atatatatatatata (SEQ ID NO:318) |
| TTTCTGGGT | TGATAAATA | | | tatatatata |
| TTTAGATTT | TATTAACCC | | | |
| GG | AG | | | |
| SARA 1 (SEQ ID NO:319) | SARA 2 (SEQ ID NO:320) | 217444 | 217492 | tctatctatctatct (SEQ ID NO:321) |
| CATGCGGG | TTCTCTAGA | | | atctatctatctatc |
| TTAATACT | GGGACAGA | | | tatctatctatctat |
| TAAT | ACG | | | ccat |
| SARA 3 (SEQ ID NO:322) | SARA 4 (SEQ ID NO:323) | 219183 | 219214 | gtttttgtttgtttgtt (SEQ ID NO:324) |
| TTTCCTGTG | GTTGCACTC | | | tgtctgtttgttttt |
| CATAGATTT | CAGCCTGG | | | |
| AC | GCG | | | |
| SARA 39 (SEQ ID NO:325) | SARA 40 (SEQ ID NO:326) | 229431 | 229467 | gagagaaagaga (SEQ ID NO:327) |
| CTGGATTTG | GTGGCCCC | | | agcaaagcagag |
| CAGCAGCC | ACAGACCCT | | | agagagagagag |
| ACT | ATC | | | a |
| SARA 33 (SEQ ID NO:328) | SARA 34 (SEQ ID NO:329) | 230749 | 230810 | cacacacacaca (SEQ ID NO:330) |
| ACAGAGTG | TGTTGGGA | | | cacacacacaca |
| AGACCCTGT | CCCAAGCA | | | cacacatacacac |
| CTG | GCAG | | | acacacatcccca |
| | | | | cacaacaacaca |
| SARA 35 (SEQ ID NO:331) | SARA 36 (SEQ ID NO:332) | 231619 | 231709 | aaaaaaaaaaga (SEQ ID NO:333) |
| CAGGTGCTT | AATACTTTC | | | gagagagaaaac |
| CAAGGTTAT | CTTCAGCAT | | | agaaaaaagaata |
| TC | TC | | | aaaagcctttttat |
| | | | | gtttttctatcttttttt |
| | | | | ctctctttcctctct |
| | | | | gctttct |
| SARA 37 (SEQ ID NO:334) | SARA 38 (SEQ ID NO:335) | 234817 | 234857 | tctgtctctctctta |
| AAGTGTATG | TTATATCCA | | | ctccctctctctcg |
| AGCCAATTC | TGTATTAGT | | | attctgtttccc |
| TG | CA | | | |
| SARA 11 (SEQ ID NO:337) | SARA 12 (SEQ ID NO:338) | 243340 | 243365 | tatatgtaagtgtgt (SEQ ID NO:339) |
| GGTCCTATG | AGACACAAA | | | gtatagatatg |
| TGGTATGAA | ATTAGGCAT | | | |
| GG | GC | | | |
| SARA 13 (SEQ ID NO:340) | SARA 14 (SEQ ID NO:341) | 245299 | 245342 | acacacacacac (SEQ ID NO:342) |
| CTTTTCAAA | ATGCCTGC | | | acacacgcacac |
| TCTCTGCAT | CTGGAAAG | | | acacacgcacac |
| GG | CTGC | | | acacacac |
| SARA 21 (SEQ ID NO:343) | SARA 22 (SEQ ID NO:344) | 249355 | 249387 | tatatatctatatgt (SEQ ID NO:345) |
| TGTCTCCCT | AATAAAACA | | | agatctatatctgt |
| AACACACTA | GAAACAATA | | | ctct |
| GG | CC | | | |
| SARA 23 (SEQ ID NO:346) | SARA 24 (SEQ ID NO:347) | 249821 | 249860 | ctttctctctcttctc (SEQ ID NO:348) |
| TGCATTTCT | GTGAAAGG | | | cttttactttatttttg |
| TCTCACAGT | GAGCAGAG | | | tccctct |
| CC | AAAG | | | |
| SARA 9 (SEQ ID NO:349) | SARA 10 (SEQ ID NO:350) | 253000 | 253044 | tctctctgtgttgtt (SEQ ID NO:351) |
| TTCTATGCC | ATCTAATAT | | | cacatgatctctct |
| TCTCTTCTT | GACAGGTG | | | ctgtgtgtgtgtgt |
| GG | TCC | | | gt |
| SARA 31 (SEQ ID NO:352) | SARA 32 (SEQ ID NO:353) | 263177 | 263211 | attttatttttattttta (SEQ ID NO:354) |
| TGCACTCCA | TTCAACACT | | | tttttatttttattttt |
| GCCTGAGC | TAAGAATGG | | | |
| GAC | GG | | | |
| SARA 5 (SEQ ID NO:355) | SARA 6 (SEQ ID NO:356) | 265833 | 265858 | tatatatatatatat (SEQ ID NO:357) |
| GGTAAGTG | AAAGGATGA | | | gtatgtatgta |
| ACAGAGTCA | CACTCAATT | | | |
| GGT | GG | | | |
| SARA 7 (SEQ ID NO:358) | SARA 8 (SEQ ID NO:359) | 266114 | 266161 | tgtgtgtgtgtgtgt (SEQ ID NO:360) |
| TAGCGGCA | CTTCTCTAC | | | gtgtgtgtgtgtgt |
| ATGTACAGC AGTTTATAA | | | gtgtgtgtgtatgt | |
| TGA | CC | | | gtgtg |
| SARA 27 (SEQ ID NO:361) | SARA 28 (SEQ ID NO:362) | 290719 | 290745 | atatacatacatat (SEQ ID NO:363) |
| TACGAAGTA | CACATAGTC | | | ataaaatatatat |
| GTTTAAAAA | TCTATATAT | | | |
| TG | TG | | | |
| SARA 29 (SEQ ID NO:364) | SARA 30 (SEQ ID NO:365) | 290427 | 290463 | gaaaagaaaaga (SEQ ID NO:366) |
| ATAAAGCCC | CTGGGGAA | | | aaagaaaagaaa |
| CAGATTTTT | CAGAGTAAA | | | gagagagaaaaa |
| G | CCC | | | g |
| SARA 47 (SEQ ID NO:367) | SARA 48 (SEQ ID NO:368) | 295275 | 295326 | gtgtgtgtgtgagt (SEQ ID NO:369) |
| ggtgttgaagcat | TCCCCTCTC | | | gtgtgtgtgtgtgt |
| aaagatg | CATTGCCTT | | | gtgtgcacgtgtgt |
| | TC | | | gtttgtgtgt |

TABLE III

| SNP POSITION | SNP and 5′ and 3′ sequence | SEQ ID NO: |
|---|---|---|
| SNP 243 | taattcttccaaaaaaaaaaaAccgtaataca | SEQ ID NO: 370 |
| SNP 1080 | gatgggcactGatgtgtttct | SEQ ID NO: 371 |
| SNP 2128 | gactccatctaaaaaaaaaaaAtatatatata | SEQ ID NO: 372 |
| SNP 6930 | agttggctttActttccttct | SEQ ID NO: 373 |
| SNP 8300 | gccctcgattAgaaatgagag | SEQ ID NO: 374 |
| SNP 9844 | ctaatcatatAtttttttgaa | SEQ ID NO: 375 |
| SNP 13809 | ggattacaggCgcacaccact | SEQ ID NO: 376 |
| SNP 20590 | tttcaacaggTagccttactt | SEQ ID NO: 377 |
| SNP 24893 | caccttatggTtgctattttt | SEQ ID NO: 378 |
| SNP 27842 | taattgttatCataattatta | SEQ ID NO: 379 |
| SNP 29938 | acaccttattCttcatgtaat | SEQ ID NO: 380 |
| SNP 34307 | tgcaactgcaCggaaactgaa | SEQ ID NO: 381 |
| SNP 41872 | ttttcttttctttTctttctctct | SEQ ID NO: 382 |
| SNP 49112 | tttcaagtcaTtttgaagtaa | SEQ ID NO: 383 |
| SNP 50661 | caagaaattaGaaaccagcca | SEQ ID NO: 384 |
| SNP 56652 | tgtgcattttTacacatgccc | SEQ ID NO: 385 |
| SNP 57187 | tgcataaaagCcttcagtaga | SEQ ID NO: 386 |
| SNP 57226 | gagagcccaaCctctctaatg | SEQ ID NO: 387 |
| SNP 57377 | tctctctttgTcttatcctcc | SEQ ID NO: 388 |
| SNP 57435 | tctcccacctCaccccagtcc | SEQ ID NO: 389 |
| SNP 57826 | tcccttccctCtctccacctc | SEQ ID NO: 390 |
| SNP 59532 | atcattagttAttagagaaat | SEQ ID NO: 391 |
| SNP 60017 | agccacatatTgtatgattct | SEQ ID NO: 392 |
| SNP 108669 | tttggtgtttNcgggagtttt | SEQ ID NO: 393 |
| SNP 109938 | tttctcttttAaaaaacagat | SEQ ID NO: 394 |
| SNP 110501 | gtagtgtggtaaaAtatctaagac | SEQ ID NO: 395 |
| SNP 110684 | gtcgaggtcaccCgtgcactgca | SEQ ID NO: 396 |
| SNP 110719 | gttcaaagccAtatcccgtga | SEQ ID NO: 397 |
| SNP 110739 | attttctagcAcagactttac | SEQ ID NO: 398 |
| SNP 114387 | aaaattaagaCattttgtttt | SEQ ID NO: 399 |
| SNP 120280 | ctaaaaatacaaaaaAttagccaggc | SEQ ID NO: 400 |
| SNP 120403 | acttcagcctGggcaacagag | SEQ ID NO: 401 |
| SNP 121010 | ctgactggtgTatttacaatc | SEQ ID NO: 402 |
| SNP 121990 | gtcatctctcAataggatgca | SEQ ID NO: 403 |
| SNP 122033 | ggaaaaacacCtgattgcttc | SEQ ID NO: 404 |
| SNP 126110 | tgaagccaacCcaccctggat | SEQ ID NO: 405 |
| SNP 127987 | acatcagtgaAggacaacact | SEQ ID NO: 406 |
| SNP 128478 | aacttttcaGtgatgcaatg | SEQ ID NO: 407 |
| SNP 132652 | accacgcttgGggaagggttt | SEQ ID NO: 408 |
| SNP 133418 | ttgtcagcatGcaaatcacca | SEQ ID NO: 409 |
| SNP 133520 | agtgcctgggGaactgctttt | SEQ ID NO: 410 |
| SNP 134514 | accaacaaatTagggtgaggg | SEQ ID NO: 411 |
| SNP 139233 | gtgtgtgtgtCtgtgtgtctg | SEQ ID NO: 412 |
| SNP 141328 | ttttcttcttCtttcctaagc | SEQ ID NO: 413 |
| SNP 143835 | ttgagggggaAgtctgggcat | SEQ ID NO: 414 |
| SNP 157313 | gcctggctaaTtttttgtatt | SEQ ID NO: 415 |
| SNP 173359 | agacatccatcCaatggaatac | SEQ ID NO: 416 |
| SNP 173984 | tcaaacttctCtgagcagtcc | SEQ ID NO: 417 |
| SNP 174036 | agatagtgctAcaaggaatga | SEQ ID NO: 418 |
| SNP 179878 | aaaaaaacacGtgaatgtaaa | SEQ ID NO: 419 |
| SNP 183361 | cacctcctctCttgcctgcca | SEQ ID NO: 420 |
| SNP 196994 | agggactgaaAattaatctac | SEQ ID NO: 421 |
| SNP 214586 | tgtctctactaaaaaaaaaaaaaaaa | SEQ ID NO: 422 |
| | aaaaaaaaaaaaaaAttacctgggt | |
| SNP 222851 | catgaggtgtTgcaccctgtg | SEQ ID NO: 423 |
| SNP 223271 | tccatttaagCggcagggttt | SEQ ID NO: 424 |
| SNP 224597 | cctgtccaagGaattcagggg | SEQ ID NO: 425 |
| SNP 224679 | actggctctaCaatagtcatg | SEQ ID NO: 426 |
| SNP 225479 | gataacaaacTcactcctgtt | SEQ ID NO: 427 |
| SNP 226412 | aaatgtgaaaTtatctcactt | SEQ ID NO: 428 |
| SNP 228418 | gagtcccaccAtctcattttt | SEQ ID NO: 429 |
| SNP 228913 | tcatctttatTgatgctaata | SEQ ID NO: 430 |
| SNP 229855 | attcgcatgcAccttacggtg | SEQ ID NO: 431 |
| SNP 230639 | ccagctactcGggagtctgag | SEQ ID NO: 432 |
| SNP 230801 | acatccccacaAaacaacaca | SEQ ID NO: 433 |
| SNP 232195 | caaacctgcaTattttgcaca | SEQ ID NO: 434 |
| SNP 232790 | gagcttgagaAaggaagcctg | SEQ ID NO: 435 |
| SNP 234071 | caggatttacAtttcaaatac | SEQ ID NO: 436 |
| SNP 234370 | atttgatcaaAcattattcta | SEQ ID NO: 437 |
| SNP 234431 | aaatcagtagTctgaataaag | SEQ ID NO: 438 |
| SNP 234996 | tcatgtgtagAtctttttgga | SEQ ID NO: 439 |
| SNP 235532 | gggaggtaggTctactttgcc | SEQ ID NO: 440 |
| SNP 235612 | atctaggttcCcagaggggaa | SEQ ID NO: 441 |
| SNP 235928 | agcacaggttGtattgggact | SEQ ID NO: 442 |
| SNP 236693 | gtgaatgcagCataggaaaga | SEQ ID NO: 443 |
| SNP 236971 | gaagaagagaGttgactaaag | SEQ ID NO: 444 |
| SNP 238558 | ttagccagggTaagaaaaaga | SEQ ID NO: 445 |
| SNP 238903 | aaaaaaaaaaTaaaggaatcc | SEQ ID NO: 446 |
| SNP 239015 | ttggtgaaggCtggtagttca | SEQ ID NO: 447 |
| SNP 239867 | aaagaattcaGaaattcataa | SEQ ID NO: 448 |
| SNP 240167 | gcttcaaccaAtaaaaatgtg | SEQ ID NO: 449 |
| SNP 240794 | tcacttttggGgtcatatatt | SEQ ID NO: 450 |
| SNP 240825 | ggacaagtgtGtattttcaat | SEQ ID NO: 451 |
| SNP 240956 | tttctcttgtGcacaaatcat | SEQ ID NO: 452 |
| SNP 241027 | agcaggagcaGagataatcta | SEQ ID NO: 453 |
| SNP 241354 | cttatctgtgaaaaaaaaaaAtgttacgagc | SEQ ID NO: 454 |
| SNP 241836 | tacattcacaCaaaaacatgc | SEQ ID NO: 455 |
| SNP 242422 | tagtagggtagGttgtatatgt | SEQ ID NO: 456 |
| SNP 242602 | aaaagtttggGagggtcattt | SEQ ID NO: 457 |
| SNP 242629 | tacctacgggGaaaatagctt | SEQ ID NO: 458 |
| SNP 242712 | taaacttgggGaggtagaaac | SEQ ID NO: 459 |
| SNP 243729 | cataaatttcAtaacttttta | SEQ ID NO: 460 |
| SNP 243917 | tgtatgcacaCttttgcattt | SEQ ID NO: 461 |
| SNP 244266 | tgtaactctgCcaatgcctga | SEQ ID NO: 462 |
| SNP 244368 | gaaaccatgcAtcatcacttc | SEQ ID NO: 463 |
| SNP 245446 | tcacatcagacCaatttgtcca | SEQ ID NO: 464 |
| SNP 245550 | taaaaaatttAaaaaaaaacc | SEQ ID NO: 465 |
| SNP 249741 | cctgggtgttTtcaataaacc | SEQ ID NO: 466 |
| SNP 250288 | taatttatgcCtttgaaaggc | SEQ ID NO: 467 |
| SNP 250513 | aaactttttgTcctcaaacct | SEQ ID NO: 468 |
| SNP 251979 | tttattctaaggGcagtgggttc | SEQ ID NO: 469 |
| SNP 252130 | tgctctccacTgctgtttaaa | SEQ ID NO: 470 |
| SNP 252881 | acaacagaaaCttctcataat | SEQ ID NO: 471 |
| SNP 253030 | tgatctctctGtgtgtgtgtg | SEQ ID NO: 472 |
| SNP 253686 | caccatcttaTttgtcataat | SEQ ID NO: 473 |
| SNP 256499 | cctgtaatctGagcactttgg | SEQ ID NO: 474 |
| SNP 256570 | gccaacatggCgaaaccctgt | SEQ ID NO: 475 |
| SNP 256654 | ggaggctgagTcatgagaatc | SEQ ID NO: 476 |
| SNP 257276 | catacccataTacaaacattc | SEQ ID NO: 477 |
| SNP 257431 | tttaaggtagActaggctaag | SEQ ID NO: 478 |
| SNP 257568 | aatttatactGtagatataga | SEQ ID NO: 479 |
| SNP 258093 | taattaaaaattttttTcatcttatta | SEQ ID NO: 480 |
| SNP 259397 | tattcacagattttTctttttaaaa | SEQ ID NO: 481 |
| SNP 259905 | ttaaaaaatcGatcagtatct | SEQ ID NO: 482 |
| SNP 260191 | ttaaataataTaaagaaccaa | SEQ ID NO: 483 |
| SNP 260961 | attgtttccaCcaattttaca | SEQ ID NO: 484 |
| SNP 262674 | cagagagtctAagatagaacc | SEQ ID NO: 485 |
| SNP 263521 | ttatatgttaAtttcttaaaa | SEQ ID NO: 486 |
| SNP 263777 | aatcaaaattccCagtggaatat | SEQ ID NO: 487 |
| SNP 263844 | tgattttcagGttcatttggc | SEQ ID NO: 488 |
| SNP 264175 | gaagcagattAttgggcttag | SEQ ID NO: 489 |
| SNP 264654 | tatatatatgTtgtacatata | SEQ ID NO: 490 |
| SNP 265508 | acaggcgcccAccatcacacc | SEQ ID NO: 491 |
| SNP 266067 | atcagaagagAtggttacact | SEQ ID NO: 492 |
| SNP 300867 | cccttgaaaaTaaggtaatgt | SEQ ID NO: 493 |
| SNP 301816 | ggtcagatagAtctgtagaaa | SEQ ID NO: 494 |
| SNP 302415 | ggttggggcaTggaaataagg | SEQ ID NO: 495 |
| SNP 302474 | ataagagatcGgggcgcagag | SEQ ID NO: 496 |
| SNP 302557 | agaagtggtcGggggtttctt | SEQ ID NO: 497 |
| SNP 302614 | aaggggttggGgtacttgccc | SEQ ID NO: 498 |
| SNP 302711 | aaacatgggtGaataatcaga | SEQ ID NO: 499 |
| SNP 303540 | ttagaagcagGtgttttgtag | SEQ ID NO: 500 |
| SNP 304319 | caaatatataCttatataata | SEQ ID NO: 501 |
| SNP 304693 | ggtggcactgTgtctcccctt | SEQ ID NO: 502 |
| SNP 304871 | atgctttgcaCcacctcccac | SEQ ID NO: 503 |
| SNP 305199 | gaatctgaccGaattgcacca | SEQ ID NO: 504 |
| SNP 305219 | aaaatatggcTggctccttct | SEQ ID NO: 505 |
| SNP 305280 | tcttcccatgTtctcacctcc | SEQ ID NO: 506 |
| SNP 305357 | ctttttcttcAtgaagtccac | SEQ ID NO: 507 |
| SNP 305715 | tatttccgtcCaccttgatga | SEQ ID NO: 508 |
| SNP 306765 | tggttaatttTtgaaatcttt | SEQ ID NO: 509 |
| SNP 306910 | aattttcattTaaaaaacctt | SEQ ID NO: 510 |
| SNP 307177 | aaatttatttActtacagttc | SEQ ID NO: 511 |
| SNP 307617 | cacacgttcaCgcttccaatg | SEQ ID NO: 512 |
| SNP 307701 | tagcaaataaTattatctact | SEQ ID NO: 513 |
| SNP 308314 | actgtgaaatGaagttttgtg | SEQ ID NO: 514 |
| SNP 308532 | gttaaatgctGtggtgtctga | SEQ ID NO: 515 |
| SNP 308852 | agaaattcaaCtgtccagatt | SEQ ID NO: 516 |
| SNP 309162 | tcattctcctCtcttatctcc | SEQ ID NO: 517 |
| SNP 309195 | cacactggggAaggctgcgaa | SEQ ID NO: 518 |
| SNP 309416 | cttgtcatacCtgagaagctc | SEQ ID NO: 519 |
| SNP 309522 | actcctgctaCatccttttag | SEQ ID NO: 520 |
| SNP 309753 | gtaaaatctgCttacctaacc | SEQ ID NO: 521 |
| SNP 310253 | tcactctaacGtggggactca | SEQ ID NO: 522 |
| SNP 310401 | atatgataaaCttttcttcct | SEQ ID NO: 523 |
| SNP 311249 | tgtctctactaaaaAtacaaaaaat | SEQ ID NO: 524 |
| SNP 314397 | aagccagtctaAccttttcatg | SEQ ID NO: 525 |
| SNP 316490 | ctataaatctcCtagaaggaag | SEQ ID NO: 526 |
| SNP 317398 | cctggatacaggGcagatgtgga | SEQ ID NO: 527 |
| SNP 318773 | aaaggagatgGtcaataggag | SEQ ID NO: 528 |
| SNP 326432 | agacgcattaGggtttggaac | SEQ ID NO: 529 |
| SNP 332250 | tttatttattTattaattttt | SEQ ID NO: 530 |
| SNP 339563 | atgaatgcagTgagaaacacg | SEQ ID NO: 531 |
| SNP 342367 | cagctttctgttTgtttgatttc | SEQ ID NO: 532 |
| SNP 343135 | agggacttggAaagtcaggct | SEQ ID NO: 533 |
| SNP 349945 | tctgtgtgtcGggtctccttt | SEQ ID NO: 534 |
| SNP 350161 | ataatcttacaAttgaatctca | SEQ ID NO: 535 |
| SNP 350578 | ccaccccccagggGtttctcactc | SEQ ID NO: 536 |
| SNP 355440 | actttattccCttgttaggct | SEQ ID NO: 537 |
| SNP 356996 | tttctcttatAccatctgttt | SEQ ID NO: 538 |
| SNP 357054 | aaaatatattTatagaaagat | SEQ ID NO: 539 |
| SNP 362429 | ttaaaactgcaAtaactccaag | SEQ ID NO: 540 |
| SNP 364707 | ttcacattctCttaggtaaag | SEQ ID NO: 541 |
| SNP 366442 | ttcacaaactTttttaactca | SEQ ID NO: 542 |
| SNP 379229 | aaaataacatAcaaggaaaaa | SEQ ID NO: 543 |
| SNP 380507 | attcagccaaAatttctgcta | SEQ ID NO: 544 |
| SNP 380660 | tcaaaaatgaAaaaacccaga | SEQ ID NO: 545 |

TABLE IV
Summary of 2q33 Sequence Information

| Feature | | Total | Ave | Std | Proportion of |
|---|---|---|---|---|---|
| Type | Number | Length | Length | Dev | Analyzed Region |
| Simple Repeats | 353 | 9604 | 27 | 27 | 2.52% |
| Complex Repeats | 368 | 60536 | 151 | 68 | 15.87% |
| Grail ORFs | 118 | 18799 | 159 | 130 | 4.93% |
| DiCTion ORFs | 70 | 17476 | 250 | 110 | 4.58% |
| Syntenic Mouse >35 bp | 70 | 8497 | 121 | 124 | — |
| Costimulatory Receptors | 3 | 62285 | — | — | 16.33% |
| (Transcribed Unit) | | | | | |
| Other Genes/Pseudogenes/EST | 17 | 15382 | — | — | 4.03% |
| Sequence Tagged Sites | 22 | 9241 | | | 2.42% |

TABLE V
Feature Table of the Human Costimulatory Receptor Region of Chromosome 2q33

| | Position | Position | | | | | Position | Position | | | |
|---|---|---|---|---|---|---|---|---|---|---|---|
| Receptor | Start | End | Size | Intron | Size | Gene/EST | Start | End | Size | Reference | Notes |
| CD28 | 42348 | 42569 | 222 | | | NADH ubiquinone | 7838 | 8329 | 491 | AF201077 | |
| 5′UTR | | | | | | oxidoreductase | | | | | |
| | | | | | | homolog | | | | | |
| CD28 | 42570 | 42621 | 52 | CD28 | 19883 | EST | 74209 | 74682 | 473 | AA311148 | from Jurkat |
| CDS-1 | | | | intron 1 | | | | | | | T-cell |
| | | | | | | | | | | | library |
| CD28 | 62505 | 62861 | 357 | GD28 | 2678 | EST | 75932 | 76379 | 447 | N20227 | from Melanocyte |
| CDS-2 | | | | intron 2 | | | | | | | library |
| CD28 | 65540 | 65664 | 125 | CD28 | 5010 | EST | 88605 | 88873 | 268 | AA663852 | from schizo |
| CDS-3 | | | | intron 3 | | | | | | | brain library |
| CD28 | 70675 | 70803 | 129 | | | EST | 93458 | 93983 | 525 | AA744591 | vicinity of |
| CDS-4 | | | | | | | | | | | multiple |
| | | | | | | | | | | | repeat elements |
| CD28 | 70804 | 73724 | 2921 | | | EST | 94424 | 94744 | 320 | H89084 | multiple EST hits |
| 3′UTR | | | | | | EST | 95762 | 96257 | 495 | AW237774 | multiple EST hits |
| CTLA4 | 203644 | 203799 | 156 | | | EST | 98855 | 99173 | 318 | L44301 | human thymus |
| 5′UTR | | | | | | | | | | | library |
| CTLA4 | 203800 | 203908 | 109 | CTLA4 | 2534 | Keratin 18 | 100130 | 101424 | 1294 | M26325, | multiple stops |
| CDS-1 | | | | intron 1 | | pseudogene | | | | #NM_000224 | |
| CTLA4 | 206443 | 206790 | 348 | CTLA4 | 444 | Nucleophosmin | 108193 | 109455 | 1262 | M26697, | multiple stops |
| CDS-2 | | | | intron 2 | | pseudogene | | | | #NM_006993 | |
| CTLA4 | 207235 | 207346 | 112 | CTLA4 | 1218 | EST homolog | 230519 | 232134 | 1615 | R91770, | vicinity of |
| CDS-3 | | | | intron 3 | | | | | | AW474005, | multiple |
| | | | | | | | | | | AI434725 | repeat elements |
| CTLA4 | 208565 | 208669 | 105 | | | EST homolog | 241762 | 242097 | 335 | AW238656, | possible distant |
| CDS-4 | | | | | | | | | | AL037926, | L1 repeat |
| | | | | | | | | | | AI905493 | |
| CTLA4 | 208670 | 209793 | 1124 | | | EST homolog | 253467 | 253534 | 67 | N73819, | vicinity of |
| 3′UTR | | | | | | | | | | AI801031, | multiple |
| | | | | | | | | | | AW079941 | repeat elements |
| | | | | | | EST homolog | 257288 | 257506 | 218 | Unigene cluster | cDNA clusters from |
| | | | | | | | | | | homolog HS 30542 | multiple tissue |
| | | | | | | | | | | | sources |
| ICOS | 272636 | 272660 | 25 | | | EST | 260890 | 261082 | 192 | AA663871 | schizo brain library |
| 5′UTR | | | | | | | | | | | |
| ICOS | 272661 | 272718 | 58 | ICOS | 18753 | EST homolog | 267282 | 269005 | 1723 | AA558770, | vicinity of |
| CDS-1 | | | | intron 1 | | | | | | AA054182, | multiple |
| | | | | | | | | | | T90825 | repeat elements |
| ICOS | 291472 | 291807 | 336 | ICOS | 685 | Endogenous | 297760 | 303099 | 5339 | AF139170, | 79% identity to |
| CDS-2 | | | | intron 2 | | retrovirus | | | | PIR A44282 | some retroviral |
| | | | | | | | | | | | elements |
| ICOS | 292493 | 292599 | 107 | ICOS | 1032 | | | | | | |
| CDS-3 | | | | intron 3 | | | | | | | |
| ICOS | 293632 | 293716 | 85 | ICOS | 1689 | | | | | | |
| CDS-4 | | | | intron 4 | | | | | | | |
| ICOS | 295406 | 295419 | 14 | | | | | | | | |
| CDS-5 | | | | | | | | | | | |
| ICOS | 295420 | 297393 | 1974 | | | | | | | | |
| 3′UTR | | | | | | | | | | | |

### EXAMPLES

The following materials and methods were used the Examples:

BAC clone selection: BAC clones were selected on the basis of positive hybridization to CTLA4, CD28 or ICOS coding sequences (Genome Systems, St. Louis, Mo.). BAC clone DNA was prepared using Concert Mega Preps BAC protocol followed by restriction endonuclease digestion of 1 ug per sample. Digested samples were electrophoresed in 7% TBE agarose gels followed by electrotransfer onto hybond membranes. Hybridization was performed against random-primed CTLA4, CD28, or ICOS cDNA probes using 0.4% White Rain Shampoo with Conditioner (Gillette, Boston, Mass.) at 55° C. for 1 hour followed by washing with 1×SSC, 1% SDS and then 0.1×SSC, 1% SDS at 55° C. until acceptable background was achieved.

BAC clone sequencing: BAC clones were shotgun cloned into pUC18 vectors followed by high throughput sequencing (Lark Technologies, Houston, Tex.). Briefly, BAC clones were sheared by spray nebulization followed by agarose fractionation and purification of 2-4 Kb and 1-2 Kb fragments. Fragments were blunt end cloned into pUC 18 SmaI site and subsequently used to generate BAC subclone libraries. Contig assembly was initially performed with GAP4 (Bonfield, J. K., et al. 1998. Nucleic Acids Res 26: 3404) and subsequent manual editing performed using Sequencher (Gene Codes, Ann Arbor, Mich.). Contig gap closure was performed by primer walk sequencing directly on BAC clones using ABI PRISM Big Dye terminator cycle sequencing chemistry and ABI PRISM 373a sequencer. Final assembly and sequence comparison was performed by alignment with Genbank sequences AC010138 (formerly H_NH0175H04), AC009965, AF225899, and AF225900.

Sequence verification: 2q33 sequence assembly was verified by BamHI, EcoRI and HindIII digests of BAC clones 22607, 22608 and 22700 and comparison with predicted restriction digest banding patterns. Although fragments were generated from 28,000 Kb to 7 bp were generated, only those ranging from greater than 2 Kb to less than 12 Kb in size were fractionated sufficiently on 0.7% agarose gels for visual analysis. The only notable discrepancy was found by the presence of a 7.7 kb BamHI restriction fragment in BAC clone 22608 not predicted by sequence data suggesting a base-miscall leading to the elimination of a BamHI site. The sequence results of BAC clone 22700 were further confirmed by restriction mapping the BAC clone using end-labeled oligonucleotide probes as hybridization probes corresponding to predicted EcoRI or SacI fragments. Blots were exposed to phosphoimage plates and processed using Fujix image plate reader and Image Reader software. Twenty-nine blot hybridizations were performed with complete accuracy to predicted DNA fragments within BAC 22700. As an external verification of contig assembly, dotplot analysis (30 bp window, 90% identity) was performed aligning 2q33 sequence with Celera Genomic Axis GA_X8WHR7H (Release 25, Celera Genomics, Rockville, Md. 20850). Resultant alignment demonstrated co-linearity between the two sequences across 300,000 bp suggesting the correct contig ordering of this genomic region.

Sequence analysis: GCG Wisconsin package 10.0 (GCG, Madison, Wis.) was used for Blast and FastA database searching. Contigs generated by sequencing were compared to protein databases using TblastN to identify potential coding sequences. After final assembly into one contig, sequences were parsed and Blast searches were performed against Genbank EST and STS databases. Positive EST hits with 80% greater were further blasted against Genbank to determine whether cDNA, Unigene or protein identity could be determined. Complex repeats and open reading frame prediction was performed by GRAIL (Genomix, Oak Ridge, Tenn.), and DiCTion (Genetics Institute, Cambridge, Mass.) under default settings. Alignment of ICOS genomic sequences was performed with GAP with a gap length penalty set to zero. The alignment output was displayed positionally using PlotSimilarity with an analysis window of 100 nucleotides. Dotplot of mouse and human ICOS genomic sequences was performed using GeneWorks (Oxford Molecular Group, Campbell, Calif.) using a window size of 20 nucleotides and 70% sequence identity cutoff. Cross species genomic sequence alignment was performed using SIM4 (Florea et al. 1998) with an F value=1.3 and word size=15. Mouse contigs with homologies greater than 35 nt in length were used in further analysis. Genomic Microarray Expression Analysis: Plasmid preparations of 864 randomly picked colonies from the BAC 22700 subclone library were used as templates for PCR amplification. PCR amplifications were carried out using modified M13 primers in 100 ml reactions containing 10 mM Tris, 1.5 mM MgCl₂ 50 mM KCl, 200 mM each dNTP, 200 nM each primer, and 1 unit Taq polymerase (Roche Molecular Biochemicals, Mannheim, Germany). PCR products were analyzed by agarose gel electrophoresis and scored for the presence of a single band resulting in 620/864 subclones yielding a robust single band. PCR products were purified using Millipore MultiScreen-FB filter plates essentially as described by the manufacturer (Millipore, Bedford, Mass.). Dried PCR products were resuspended in 5M sodium thiocyanate and spotted in duplicate onto Type VI slides (Molecular Dynamics, Sunnyvale, Calif.) using a GenII arrayer (Molecular Dynamics, Sunnyvale, Calif.). Probes were prepared by including Cy3 or Cy5 labeled dCTP (Amersham Pharmacia Biotech, Piscataway, N.J.) in oligo-(dT) primed first-strand cDNA synthesis reactions from 10 mg total RNA essentially as described (Schena et al. 1996). Hybridizations were carried out at 42° C. for 16 hrs in buffer containing 50% formamide, 5×SSC, 0.1% SDS and 100 mg/ml human COT-1 DNA (Life Technologies, Rockville, Md.). The arrays were washed at room temperature once in 1×SSC, 0.2% SDS for 5 min, and twice in 0.1×SSC, 0.2% SDS for 10 min then rinsed in water and dried with compressed nitrogen. Scanning was carried out using a ScanArray 5000 confocal laser scanner (GSI Lumonics, Waltham, Mass.) and quantitated using ArrayVision 4.0 (Imaging Research, Inc, St. Catharines, ON, Canada). Data from replicate spots on three arrays were combined by taking the average of the log transformed ratio. Differential upregulation was defined as 1.5 fold induction in at least 5/6 measurements and having a total signal intensity above a background threshold (1,000 for Cy3+Cy5 on BAC37 reference control.)

Microsatellelite Polymorphism Analysis: Human donor placental and peripheral blood DNA were used as amplification templates. Single members of oligonucleotide pairs were end-labelled with gamma-³²P-ATP using T4 polynucleotide kinase (New England Biolabs, Beverly, Mass.) followed by purification through G25 spin columns. Fifteen ul PCR reactions were performed using Platinum Taq (Life Technologies) according to manufacturer's protocol using 5 pM of each primer and cycled 30 times with the parameters: 95° C. 1 min. 60° C. 1 min., and 72° C. 1 min. Amplified microsatellite DNA was fractionated on Novex QuickPoint Sequencing gels (Invitrogen, Carlsbad, Calif.). Microsatellite amplification primer pairs used included: SARA 1: CATGCGGGTT AATACTTAAT (SEQ ID NO:319), SARA2: TTCTCTAGAG GGACAGAACG (SEQ ID NO:320); SARA 31: TGCACTCCAG CCTGAGCGAC (SEQ ID NO: 352), SARA 32: TTCAACACTT AAGAATGGGG (SEQ ID NO:353); SARA 43: TATTTCTCCT CTTTCACTGG, TGACCTGAAA TAAACATAGA; Sara 47: GGTGTTGAAG CATAAAGATG (SEQ ID NO: 367), TCCCCTCTCC ATTGCCTTTC (SEQ ID NO:368); CTLA4 3′UTR: TAGCCAGTGA TGCTAAAGGT TG (SEQ ID NO: 548), AACATACGTG GCTCTATGCA CA (SEQ ID NO:549; position start: 209,177 position end 209,216) ; ICOS 3′UTR retrovirus: GCAAAGAATA AACATTTGAT ATTCAGC (SEQ ID NO:550), CCCCCCTTTG AATGTAATTT TCCTTTACG (SEQ ID NO:551) and having start and end positions at 297,760 and 303,099, respectively.

### Example 1

Physical Mapping, Genomic Sequencing and Assembly of 2q33 Costimulatory Receptor Cluster.

To determine the degree of overlap and distance between CTLA4, CD28, and ICOS, 6 independent BAC clones were isolated by hybridization to costimulatory receptor cDNA probes. Of the 6 separate BAC clones, two exhibited hybridization with CD28, two with CTLA4, one with ICOS, and one with both CTLA4 and ICOS. Each BAC clone was end-sequenced and PCR primer sets were designed to examine BAC clone overlap. Overlapping PCR sets were detected between BAC clones resulting in a hypothetical map of the costimulatory receptor region clustered in the order of CD28, CTLA4, and ICOS. Three fold shotgun sequencing of clone 22700 library resulted in the generation of 1,151 end reads collapsing into 70 contigs spanning approximately 170 kb. Two fold sequencing of clone 22606 and 22608 library generated 960 sequences collapsing into 107 contigs spanning 130 kb, and 960 sequences collapsing into 111 contigs spanning 107 kb, respectively. Mouse BAC clone 23114 was sequenced two-fold generating 767 end read sequences collapsing into 143 contigs spanning 131 kb. Big-Dye primer sequencing was performed directly on BAC clone DNA using primers designed from the sequences flanking gapped sites to close selected gaps in sequence.

BAC clones were end sequenced and PCR primer sets designed specific to each BAC end. Amplification of each BAC clone with the complete set of PCR primers resulted in amplification patterns corresponding to the genomic organization of the costimulatory receptors. Starting and ending positions based on subsequent sequence data are indicated for each BAC clone (N. D.=Not determined): BAC 22606 (N. D.−66,887), BAC 22607 (N. D.−167,094), BAC 22701 (74,706-278,563), BAC 22699 (84,599-239,485), BAC 22700 (119,296-300,949), BAC 22608 (233,866-381,403).

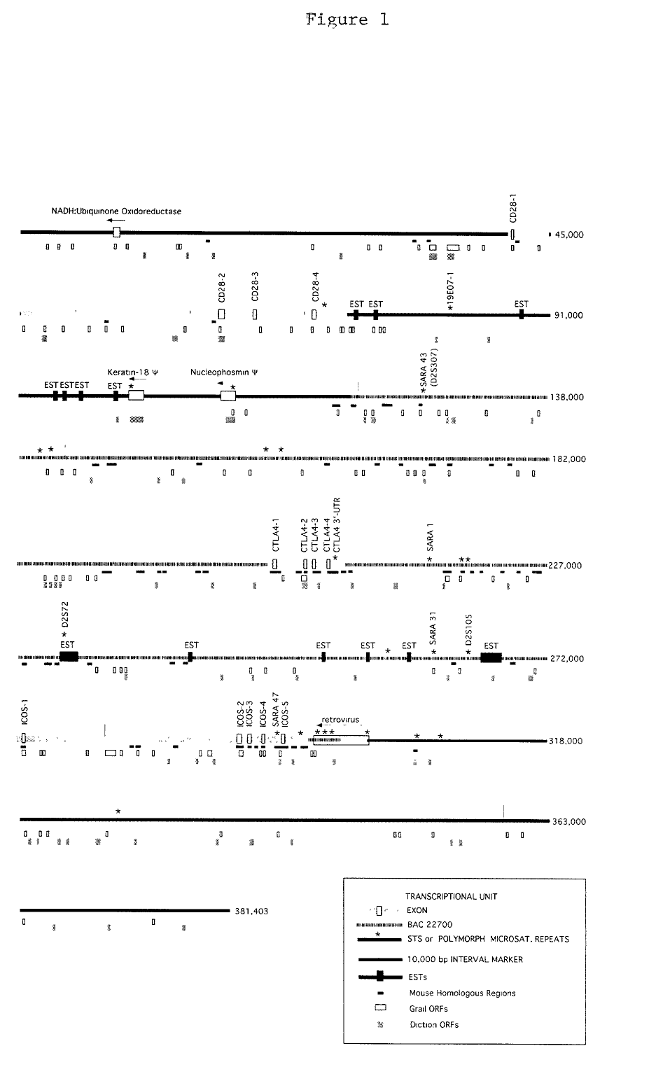

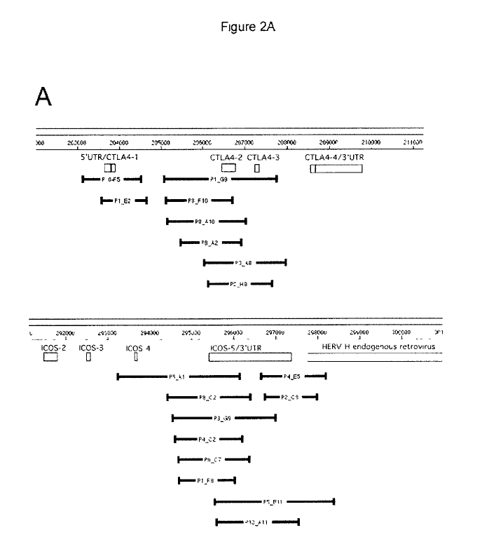

When necessary, overlaps to publicly available genomic data were used to position contigs, especially PAC clone p61e2 (Accession #AF225900), bridging the 52,408 bp gap between nt. 66,888 to nt. 119,295. Merging BAC clones with existing sequences resulted in one contiguous sequence of 381,403 bp initiating 42,570 bp upstream of CD28, and ending 85,985 bp downstream of ICOS (FIG. 1).

### Example 2

Genomic Organization of 2q33 Genes, Homologs, STS and ESTs.

Twenty potential protein coding elements were identified within the 381 kb costimulatory receptor region with sequences exhibiting either identity to or homology with known genes or ESTs (Table IV and Table V): NADH: ubiquinone oxidoreductase homolog, CD28 (NM_{—}006139), keratin-18 pseudogene, nucleophosmin pseudogene, CTLA4 (NM_{—}005214), Unigene HS.30542 homolog, ESTs, ICOS (Genseq #V53199), and an element similar to many human endogenous retrovirus type H with associated 5′ and 3′ LTR (RTLV-H2, M18048; amongst others). Based on a recent mapping study of 2q31-33, the three receptor loci within this region are situated on the chromosome with CD28 being the most centromeric and markers, now known to be near ICOS, being the most telomeric (Deng, Z., et al. 2000. Am J Hum Genet 67:737). In addition, 22 STS (sequence tag sites) were identified upon BLAST search of this compiled region of 2q33, of which 4 correlated to endogenous retroviral sequence. The commonly used genetic markers for 2q33, D2S307 (SARA 43), D2S72, D2S105, and 19E07-1 were contained within the sequence presented here. Because HERV-H elements are found in ˜1000 copies in the genome, it remains to be determined if these 4 STS are specific for the element described here. Based on human ICOS cDNA sequence data, the organization of the ICOS locus was determined to be comprised of 5 coding sequences spanning 22,758 bp from the initiation codon of exon 1 to the termination codon of exon 5, unlike the 4 exon structure of both the CTLA4 and CD28 genes. ICOS exon 5 encoded the smallest coding sequence, represented by only 4 amino acids [(D)-V-T-L] followed by a stop codon. In other respects, exons 1-4 parallel the genomic organization of CTLA4 and CD28 with exon 1 encoding the leader sequence, exon 2 encoding the extracellular Ig-V like domain, exon 3 encoding the transmembrane domain and exon 4 and 5 encoding the cytoplasmic domain. All three costimulatory receptors shared similar pattern of intron size distribution in which intron 1>intron 3>intron 2. ICOS appeared to be more similar in genomic organization to CD28, with ICOS intron 1 spanning 18.7 kb compared to CD28 intron 1 spanning 19.9 kb, versus CTLA4 intron 1 spanning 2.5 kb.

### Example 3

Computer Assisted Prediction of Open Reading Frames.

The 381 Kb costimulatory receptor locus was analyzed by the open reading frame prediction programs DiCTion and GRAIL to assess the potential of other sequences in this region to encode gene products (FIG. 1, Table IV). DiCTion analysis of the costimulatory receptor region resulted in the prediction of 70 ORFs with a cumulative length of 17476 bp, of which 5 ORFs represented repetitive Alu sequences. Coding sequences representing CD28 exon 2 and CTLA4 exon 2, keratin-18 and nucleophosmin pseudogenes were predicted by DiCTion. DiCTion did not predict sequences encoding ICOS. Of the remaining ORFs, two were localized to intron 1 of CD28, and single ORFs were predicted in intron 3 of both CTLA4 and ICOS receptor loci. Assuming that the predicted intronic ORFs are false positives, these results suggest that up to 56 potential DiCTion ORFs remain in this region of 381 kb. GRAIL analysis generated more potential ORFs than DiCTion, with a total of 118 segments and a cumulative length of 18,799 bp (Table IV). GRAIL predicted some open reading frames containing CD28 (CDS-1, CDS-2, CDS-4), CTLA4 (CDS-2), and ICOS (CDS-1, CDS-2, CDS-4), however, neither GRAIL or DiCTion were successful in predicting the complete set of exonic sequences from any receptor and moreover, both programs predicted ORFs in known intronic sequences. For example, in the CD28 intron 1, GRAIL predicted 8 ORFs while DiCTion predicted 1 ORF. Although it has been reported that CD28 may be expressed as alternatively spliced products (Lee et al. 1990. J Immunol 145: 344-52), it has not been demonstrated that intronic sequences described here contribute to the final products of known isoform variants. When DiCTion and GRAIL outputs were compared, 13 predicted open reading frames were found in common to both. Of these, three correspond to the known sequences CD28 CDS-2, CTLA4 CDS-2 and EST M26697.

### Example 4

Genomic Microarray Expression Analysis (GMEA).

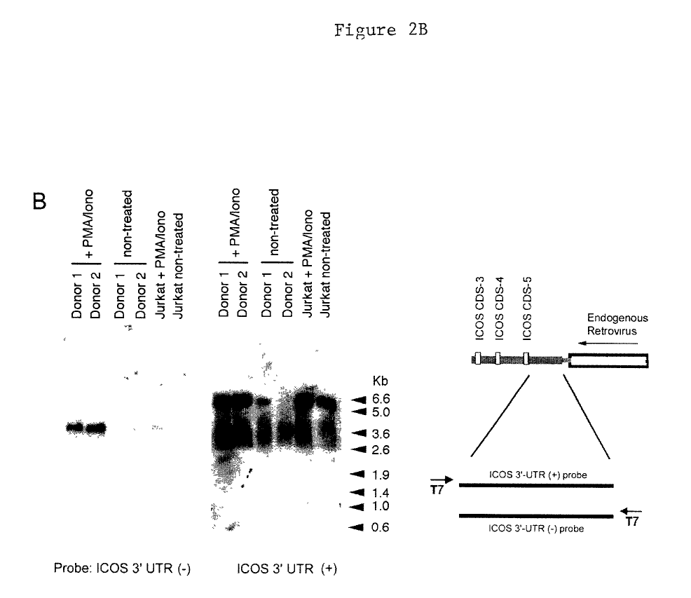

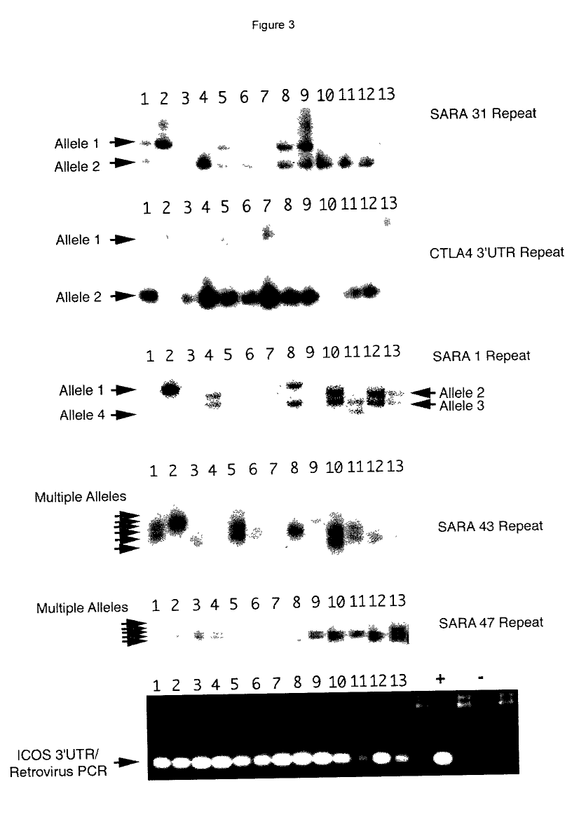

To examine whether differentially transcribed genes within this genomic region could be detected, the sequenced BAC 22700 subclone library collection was interrogated by genomic microarray expression analysis. The previously sequenced plasmid library DNA samples were amplified by PCR, the amplified DNA products were spotted onto glass slides, and hybridization was performed with total RNA from either non-stimulated or PMA-ionomycin treated CD4+ T-cells. Of the starting 864 plasmid subclones, 620 amplified products were recovered and analyzed, resulting in 18 clones showing differential hybridization in 5 out of 6 replicate experiments (3 slides each with duplicate spots). Eight clones corresponded to sequences within the CTLA4 locus, 7 clones corresponded only to the ICOS 3′ UTR and 3 clones corresponded to both ICOS 3′ UTR and endogenous retroviral sequences immediately 3′ of ICOS (FIG. 2A). It must be noted that hybridization of cDNA against genomic DNA would preferentially occur between target sequences of longer length (exon 2 and 3′ UTR of CTLA4 and ICOS); thus the degree of hybridization to microarrayed spots containing only short CDS flanked by non-differentially expressing intronic sequences could be lower. Indeed, the differential hybridization detected to ICOS was to the region corresponding to the longest transcribed unit, the 2 kb 3′ UTR. Most importantly, no clones other than CTLA4, ICOS and retrovirus immediately downstream of ICOS were found to be induced suggesting that the stringency of the experimental conditions used in this study was sufficient for detecting transcriptionally induced genes while effectively eliminating non-specific background hybridization generated by genomic and plasmid DNA.

To determine whether hybridization to ICOS and retroviral sequences reflected transcription from the ICOS promoter or whether this differential signal reflected transcripts from the endogenous retrovirus proximal to the ICOS locus, RNA blots were performed to determine transcript orientation from this region. In order to rule out cross hybridization to repetitive sequences, blast search was performed using ICOS 3′ UTR sequences adjacent to the endogenous retrovirus. No repetitive DNA was detected, and hence, this sequence was subcloned in both orientations into separate T7-promoter bearing vectors to generate strand-specific radiolabeled probes. RNA from two donor CD4+ T-cells and Jurkat T-cell line preparations, cultured either in the presence or the absence of PMA-ionomycin activation, were fractionated, blotted and hybridized to either the ICOS 3′ UTR sense or anti-sense probe (FIG. 2B). With the ICOS anti-sense probe, a clear hybridization signal was observed for activated samples but not for non-activated samples. Hybridization with ICOS sense probe also revealed two regions of clear hybridization signals in all samples examined; one discrete band at approximately 6.5 kb and one non-discrete band at ˜3-4 kb. These results strongly suggest that the retroviral LTR promoters 3′ of ICOS are transcriptionally active and are responsive to cell activation. The 6 kb band appeared to be preferentially induced on activated CD4+ T-cells while being constitutively expressed in both Jurkat cells samples. The 3-4 kb band appeared to be expressed in all samples examined regardless of activation state. Because these retroviral transcripts may be derived from either the 5′ LTR or the 3′ LTR viral promoter, at least two potential sets of transcripts may be detected. With the presence of 8 canonical polyadenylation signals (AATAAA) within the 7.5 kb upstream from the ICOS 3′ UTR, it is not possible to correlate promoter activity with observed transcript size at this time.

### Example 5

Analysis of Microsatellite Polymorphisms.

Polymorphisms in the 3′ UTR of CTLA4 have been linked to a number of autoimmune genetic diseases. To identify additional markers in this region that may also serve to refine the associations between genetic diseases and the costimulatory receptor region of 2q33, 25 microsatellite repeat sequences in the BAC 22700 clone were analyzed for the presence of repeat unit polymorphisms. Genomic DNA PCR amplification of 13 individuals revealed 4 microsatellites, corresponding to di-, tri- and hexanucleotide repeats, that demonstrated allelic polymorphisms upon analysis by denaturing acrylamide gel electrophoresis (FIG. 3). Of the 4 polymorphic microsatellite repeats examined, repeat SARA 31(nt. 263,177-263,211; [ATTTTTT]n6) was represented by 2 alleles, repeat SARA 1(nt. 217,444-217,492; [TCTA]n12) was represented by 4 alleles, while SARA 43 (nt. 125,845-125,892 [GT]n24, homologous to sequences within D2S307) and SARA 47 (nt. 295,275-295,326; [GT]n15) appeared to be highly polymorphic with at least 6 different alleles within 13 individuals examined. Analysis of the 13 individuals for the polymorphisms associated with the known CTLA4 3′ UTR (nt. 209,177-209,216; [AT]n40) microsatellite repeat demonstrated 2 alleles. Compilation and comparison of the 4 polymorphic microsatellite alleles found in these individuals revealed no shared allelic combination, indicating that this set of 4 polymorphic markers may be effectively applied to the high resolution discrimination of genetic associations of disease states linked to the costimulatory receptor region. For a positive amplification control, a primer set was used corresponding to nt. 297,362 to 297,388 (forward primer) and 297,934 to 297,907 (reverse primer) corresponding to the 3′ UTR of ICOS and to the 3′ LTR of the HERV-H. Amplification of the 13 individuals with this set of primers resulted in a single predicted band at ˜400 bp indicating the presence of this segment of DNA across the panel examined.

### Example 6

Cross Species Comparison of ICOS.

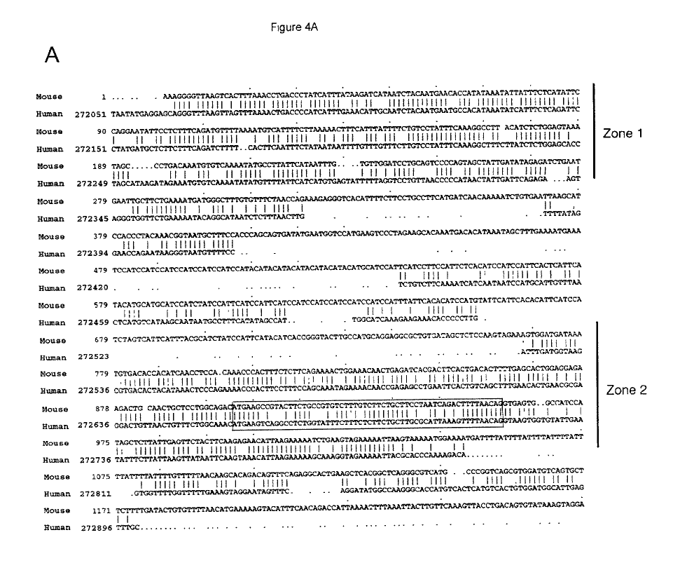

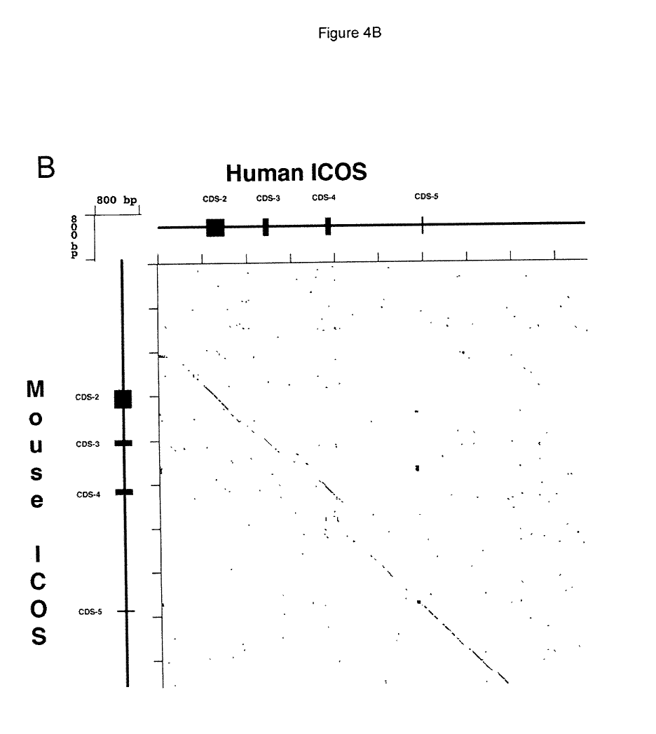

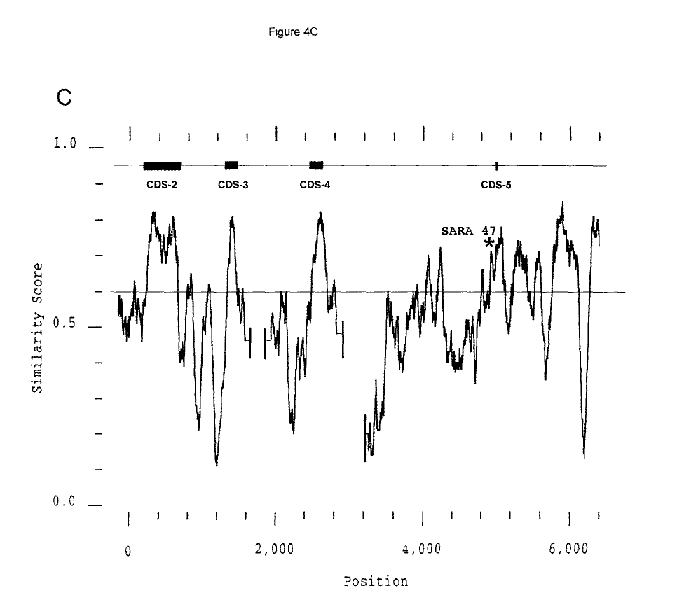

The generation of the complete sequence for the human ICOS locus along with the partial sequencing of the mouse ICOS locus allowed the cross species comparison of genomic coding and non-coding sequences in this region (FIGS. 4A, B, C). Limited gap closure of the mouse ICOS locus by primer walking resulted in the assembly of one contiguous sequence spanning CDS-2 to CDS-5 and flanked by 2265 bp of intron-1 and 1415 bp of 3′ untranslated/genomic DNA. Dotplot comparison analysis of the human genomic region was performed with the syntenic genomic region from mouse starting from 2265 bp upstream of mouse CDS-2 to 1414 bp downstream from mouse CDS-5 (FIG. 4B). Allowing for gaps, diagonals representing a minimum of 60% sequence identity were clearly observed in this aligned region; most notably, a diagonal was detected extending 3′ of CDS-5 for 2.4 Kb. A similarity plot of the gap-corrected sequence alignment of this region resulted in approximately 60% sequence identity over 6.4 kb of aligned sequence. The highest peaks of sequence similarity (˜80% identity) were clearly detected for CDS-2, CDS-3, CDS-4 and CDS-5. Intron 2 and intron 3 had lower similarity score (˜45%) owing to the presence of gaps formed by the alignment process. Gaps in alignment represented by valleys (<30% identity) were generally comprised of repetitive sequences presented in only one species. Seven peaks of high sequence identity (>70%) were found in non-coding regions of intron 4 and the 3′ UTR region starting from 1 kb upstream to 2.4 kb downstream of CDS-5. The sequence conservation in the ICOS intron-4 was especially striking, as evidenced by the presence of the SARA 47 microsatellite in both mouse and human sequences. The SARA 47 (GT)n24 intron 4 microsatellite repeat was located 88 bp 5′ of human ICOS exon 5, while a similar (GT)n48 intron 4 microsatellite repeat was discovered 66 bp 5′ of mouse ICOS exon 5.

Sequences flanking ICOS CDS-1 revealed two zones of high similarity between mouse and human genomic DNA (FIG. 4A). The first zone of high sequence identity was a 317 bp region with 72% sequence identity to mouse sequences located 276 bp upstream from initiation methionine at nt 272,661. The second zone was a 269 bp region with 75% sequence identity immediately flanking and including CDS-1, starting from 134 bp upstream of the initiation methionine to 75 bp downstream from the start of intron 1. The intervening gap (human=143 bp, mouse=448 bp) between zone 1 and zone 2 was due to a G-deficient tract of DNA unique to mouse sequence and populated with numerous low complexity TCCA, TACA and TTCA repeats. Assuming that transcriptional control regions are conserved between mouse and humans, it is likely that sequences in either zone 1 or zone 2 are responsible for transcriptional control of ICOS expression. The full-length human ICOS cDNA (Genseq #V53199) reveals 25 bp of 5′ UTR prior to initiation codon, however, whether this cDNA clone represents the actual transcription start site remains to be determined. Neither mouse or human ICOS zone 2 contains the conventional TATA promoter motif, suggesting that transcriptional start site is likely to be in zone 1 which contains multiple TATA sites. Analysis for conserved transcription factor binding sites located in both zone 1 and zone 2 by the publicly available Transfac database search revealed no T-cell specific control elements shared between mouse and human sequences. A single potential NFAT-1 site was found in mouse zone 1 along with numerous non-T cell specific sites (e.g. AP-1, AP-2, Pu.1, GATA-1, c-Jun, Gal4 and others).

The extent of sequence conservation within the intergenic region encompassing CTLA4 and ICOS receptors was examined by a comparative genomic survey of a 2× sequenced syntenic mouse BAC clone comprising 143 non-contiguous sequences aligned to the repeat-masked (DUST) human 381 kb sequence using SIM4. Of regions greater than 34 bp in length, 71 alignments were found with identity scores averaging 81%. When human sequences between nt 100,000 and 301,000 were examined, repetitive sequences comprised 36,621 bp, leaving a total of 164,379 bp of potential structural or transcribed DNA. Within this region, SIM4 mouse homologies totaled 8,531 bp theoretically corresponding to roughly 5% of the CTLA4/ICOS region. Given the limited degree of mouse BAC clone sequence coverage, only 131 kb of data was generated with the potential for an additional missing 28 kb in “unfilled” gaps, leaving the sequence determination of the syntenic mouse region be approximately 80% complete. Based on the 5% homology estimated between mouse genomic DNA syntenic and shared with human BAC clone 22700, it is not likely that extensive sequence similarities span the intergenic region between CTLA4 and ICOS, but rather, similarities are comprised of smaller stretches of homologous DNA within this region. It remains to be determined whether these stretches of homologous genomic DNA are involved with transcriptional control or whether they encode other peptide domains common to both species.

Equivalents

Those skilled in the art will recognize, or be able to ascertain using no more than routine experimentation, many equivalents to the specific embodiments of the invention described herein. Such equivalents are intended to be encompassed by the following claims.

[image]

[image]

[image]

[image]

[image]

[image]

[image]

[image]

[image]

[image]

[image]

[image]

[image]

[image]

What is claimed is:

1. A method for determining the predisposition of a human subject to develop autoimmune disease, said method comprising detecting at least one polymorphic microsatellite repeat (PMR) in the human costimulatory receptor gene locus, wherein the PMR sequence is not an hR2 sequence, to thereby determine the predisposition of a human subject to develop autoimmune disease.

2. The method of claim 1, wherein a PMR sequence is selected from the group consisting of SEQ ID Nos.: 3, 6, 9, 12, 15, 18, 21, 24, 27, 30, 33, 36, 39, 42, 44, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168,171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 321, 324, 327, 330, 333, 336, 339, 342, 345, 348, 351, 354, 357, 360, 363, 366, and 369.

3. The method of claim 1, wherein a PMR sequence is selected from the group consisting of SEQ ID Nos.: 303, 306, 309, 312, 315, 321, 324, 327, 330, 333, 336, 339, 342, 345, 348, 351, 354, 357, 360, 363, 366, and 369

4. The method of claim 2 wherein the autoimmune disease is selected from the group consisting of: insulin-dependent diabetes mellitus (IDDM), Addison's disease, Graves' disease, autoimmune hypothyroidism, myasthenia gravis, thymoma, lupus, thyroiditis, postpartum thyroiditis, rheumatoid arthritis, Hashimoto's disease, coeliac disease and leprosy.

5. The method of claim 2, wherein the step of detecting is performed using a polymerase chain reaction (PCR) employing a first and second primer.

6. The method of claim 5, wherein the first or second primer comprises a sequence selected from the group consisting of SEQ ID Nos.: 301, 302, 304, 305, 307, 308, 310, 311, 313, 314, 316, 317, 319, 320, 322, 323, 325, 326, 328, 329, 331, 332, 334, 335, 337, 338, 340, 341, 343, 344, 346, 347, 349, 350, 352, 353, 355, 356, 358, 359, 361, 362, 364, 365, 367, and 368.

7. A method for determining the predisposition of a human subject to autoimmune disease, said method comprising detecting an hR1 PMR sequence to thereby determine the predisposition of a human subject to autoimmune disease.

8. The method of claim 7, wherein the autoimmune disease is selected from the group consisting of insulin-dependent diabetes mellitus (IDDM), Addison's disease, Graves' disease, autoimmune hypothyroidism, myasthenia gravis, thymoma, lupus, thyroiditis, postpartum thyroiditis, rheumatoid arthritis, Hashimoto's disease, coeliac disease and leprosy.

9. The method of claim 7 wherein said detecting is performed using PCR employing a first and second primer.

10. A method for determining the polymorphic variant or subtype of a PMR sequence in the costimulatory receptor locus in a human subject, said method comprising detecting at least one polymorphic microsatellite repeat (PMR) in the human costimulatory receptor gene locus, wherein the PMR sequence is not an hR2 sequence to thereby determine the polymorphic variant or subtype of a PMR sequence in the costimulatory receptor locus in a human subject.

11. The method of claim 10, wherein a PMR sequence is selected from the group consisting of SEQ ID Nos.: 3, 6, 9, 12, 15, 18, 21, 24, 27, 30, 33, 36, 39, 42, 44, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168,171, 174, 177, 180, 183, 186, 189, 192, 195, 198, 201, 204, 207, 210, 213, 216, 219, 222, 225, 228, 231, 234, 237, 240, 243, 246, 249, 252, 255, 258, 261, 264, 267, 270, 273, 276, 279, 282, 285, 288, 291, 294, 297, 300, 303, 306, 309, 312, 315, 321, 324, 327, 330, 333, 336, 339, 342, 345, 348, 351, 354, 357, 360, 363, 366, and 369.

12. The method of claim 11, wherein the step of detecting is performed using PCR employing a first and second primer.

13. A PCR primer capable of amplifying a PMR sequence in the costimulatory receptor locus of a human subject, wherein the primer comprises a nucleotide sequence selected from the group consisting of: SEQ ID NO: 301, 302, 304, 305, 307, 308, 310, 311, 313, 314, 316, 317, 319, 320, 322, 323, 325, 326, 328, 329, 331, 332, 334, 335, 337, 338, 340, 341, 343, 344, 346, 347, 349, 350, 352, 353, 355, 356, 358, 359, 361, 362, 364, 365, 367, and 368.

14. A method for determining the predisposition of a human subject to develop autoimmune disease, said method comprising detecting at least one single nucleotide polymorphism (SNP) in the human costimulatory receptor gene locus, to thereby determine the predisposition of a human subject to develop autoimmune disease.
